(19) United States
(12) Patent Application Publication
(10) Pub. No.: US 2023/0374483 A1
(43) Pub. Date: Nov. 23, 2023

(54) MODIFIED HEXOSAMINIDASE AND USES THEREOF

(71) Applicant: Regents of the University of Minnesota, Minneapolis, MN (US)

(72) Inventors: Li Ou, Eden Prairie, MN (US); Chester B. Whitley, Brooklyn Park, MN (US)

(21) Appl. No.: 18/004,605

(22) PCT Filed: Jul. 8, 2021
(86) PCT No.: PCT/US2021/040820

§ 371 Date: Jan. 6, 2023

Related U.S. Application Data
(60) Provisional application No. 63/049,433, filed on Jul. 8, 2020.
(60) Provisional application No. 63/138,662, filed on Jan. 18, 2021.

(51) Int. Cl. C12N 9/24; A61P 25/00; C07K 14/775; C12N 15/86

(52) U.S. Cl. CPC C12N 9/2402; A61P 25/00; C07K 14/775; C12N 15/86; C12Y 302/01052; C07K 2319/10

(57) ABSTRACT

The disclosure provides for vectors encoding a fusion polypeptide comprising a modified hexosaminidase beta-subunit that forms homodimers, a linker and an ApoE peptide that allows for transport across the blood brain barrier, the encoded polypeptide, and methods of using the vector or polypeptide.

### CROSS-REFERENCE TO RELATED APPLICATIONS

This application is a U.S. national stage application filing under 35 U.S.C. 371 from International Application No. PCT/US2021/040820, filed Jul. 8, 2021, and published as WO 2022/011099 A1 on Jan. 13, 2022, which claims the benefit of the filing date of U.S. application No. 63/049,433, filed on Jul. 8, 2020, and U.S. application No. 63/138,662, filed on Jan. 18, 2021, the disclosures of which are incorporated by reference herein.

### INCORPORATION BY REFERENCE OF SEQUENCE LISTING

A Sequence Listing is provided herewith as a text file, “2344885.txt” created on Jun. 20, 2023 and having a size of 26,351 bytes. The contents of the text file are incorporated by reference herein in their entirety.

### BACKGROUND

GM2-gangliosidoses, including Sandhoff disease and Tay-Sachs disease, are genetic disorders causing severe neurological diseases and premature death. GM2-gangliosidoses result from deficiency of a lysosomal enzyme β-hexosaminidase A and subsequent accumulation of GM2 gangliosides. Genetic changes in HEXA, encoding the Hex α subunit, or HEXB, encoding the Hex β subunit, causes Tay-Sachs and Sandhoff disease, respectively. Only the Hex isozyme A (αβ heterodimer), but not isozyme S (αα) or B (ββ), can degrade GM2 gangliosides. Previously, a HEXM construct was developed by amino acid substitutions in the Hex a subunit (Tropak et al., 2016). The HEXM sequence encodes a subunit that can form a homodimer to degrade both Tay-Sachs disease and Sandhoff disease. Similarly, a modified HEXB sequence (designated as Mod2B) was developed by amino acid substitutions in the Hex β subunit (Kitakaze et al., 2016). The substitutions are as follows: RQNK 312-315 GSEP, LDS 316-318 SGT, and DL 452-453 NR. This Mod2B sequence encodes a subunit that can form a homodimer to treat both Tay-Sachs disease and Sandhoff disease.

### SUMMARY

Since many lysosomal diseases lead to severe neuropathology, transgene delivery across the blood brain barrier (BBB) is a challenge for gene therapy. The BBB restricts the entry of most macromolecules into the brain except for those through receptor-mediated transcytosis mechanism. As disclosed herein, a hexosaminidase-beta, e.g., HEXB (Mod2B), encoding nucleotide sequence was altered to improve transgene expression, e.g., by altering codon usage, and linked to nucleic acid sequences that encode a targeting peptide. For example, the nucleotide sequence of Mod2B, e.g., SEQ ID NO:1, was altered via codon optimization through the web server from Integrated DNA Technologies (https:/www.idtdnaopt) to yield, in one embodiment, a nucleotide sequence termed HEXX (SEQ ID NO:2) which encodes Mod2B. The HEXX nucleotide sequence was linked to nucleic acid sequences that encode a targeting peptide via a linker. In one embodiment, a nucleic acid sequence encoding a peptide that facilitates transcytosis or transport from vasculature to the brain is employed for targeting. Low-density lipoprotein receptor (LDLR) and LDL receptor-related protein 1 (LRP1) receptor are abundantly expressed on the BBB (Ueno et al., 2005), and bind and facilitate transcytosis of apolipoproteins. In one embodiment, the targeting peptide is an apolipoprotein peptide that allows for passage of the encoded fusion protein across the BBB. In one embodiment, the targeting peptide comprises a portion of ApoE which facilitates transcytosis across brain endothelial cells via LDLR receptors, LRP1 receptors, and/or M6P receptors.

In one embodiment, a vector, e.g., a plasmid or a viral vector, is provided that encodes a fusion of a lysosomal storage enzyme, e.g., hexosaminidase, linked to a peptide that enhances delivery from the blood to the brain. In one embodiment, a viral vector encoding the fusion protein is provided. In one embodiment, the viral vector may be an adenovirus vector, an adeno-associated virus (AAV) vector, a retroviral vector or a lentivirus vector. In one embodiment the vector is delivered along with a gene editing system. In one embodiment, initial intended a viral vector such as a recombinant AAV vector, as part of a gene editing system, is employed. In one embodiment, the vector is an AAV vector, e.g., non-integrating AAV, that is administered, e.g., systemically. In one embodiment, the vector is a lentiviral vector that, in one embodiment, is introduced to cells such as CD34 (bone marrow and hematopoietic stem cells) cells ex vivo, and those cells are administered to a mammal. In one embodiment, the vector is a plasmid such as a Sleeping Beauty transposon. In one embodiment, the vector is part of a nanoparticulate system having DNA or mRNA, or any type of system that can result in protein expression in the body, or even in vitro (mammalian cells such as Chinese hamster ovary cells, or plant cells such as tobacco cells, or carrot cell), to make an injectable “enzyme replacement drug”.

In one embodiment, the disclosure provides a nucleic acid vector encoding a fusion polypeptide comprising a modified hexosaminidase beta-subunit that forms homodimers, a linker and an ApoE peptide that allows for transport across the blood brain barrier. In one embodiment the vector is a plasmid. In one embodiment, the vector is a viral vector. In one embodiment, the vector encodes a multimer of the ApoE peptide. In one embodiment, the hexosaminidase beta- subunit is encoded by SEQ ID NO:2. In one embodiment, the vector comprises a nucleotide sequence having at least 80% nucleic acid sequence identity to SEQ ID NO:2. In one embodiment, the linker comprises X₁DX₂X₃E (SEQ ID NO:10), wherein X₁, X₂ or X₃ individually is I, L, V, A or G. In one embodiment, the linker comprises X₁X₄X₂X₃X₅, wherein X₄ or X₅ individually is D, E, R, K, N or Q. In one embodiment, the linker comprises IDILE. In one embodiment, the linker comprises X₆X₇X₈X₉X₁₀ (SEQ ID NO:11), wherein each of X₆, X₇, X₈ and X₉ individually is I, L, V, A or G and wherein X₁₀ is S or T or X₆X₇X₁₀, X₆X₁₀X₁₀. In one embodiment, the linker comprises GGGGS (SEQ ID NO:12), GGS, GSS, or GSSSSSS (SEQ ID NO:13). In one embodiment, the linker comprises LGGGGSGGGGSGGGGSGGGGS (SEQ ID NO:14). In one embodiment, the ApoE peptide comprises X₁₁X₁₂X₁₃X₁₄X₁₅X₁₆X₁₇X₁₈X₁₉ (SEQ ID NO:15), wherein each of X₁₁, X₁₄, X₁₅, X₁₈, and X₁₉ individually is I, L, V, A or G, and wherein each of X₁₂, X₁₃, X₁₅ X₁₆, and X₁₇ is R, K or H. In one embodiment, the linker comprises (GGGGS)n (SEQ ID NO:16), (GGGS)n, (GS)n, GSAGSAAGSGEF (SEQ ID NO:17), or (PGPG)n, where n is 0 to 20, e.g., n is 1, 2, 3, 4 5, 6 7, 8, 9, 10 or 15. In one embodiment, the ApoE peptide comprises LRKLRKRLL (SEQ ID NO:18). In one embodiment, the vector comprises an adeno-associated virus vector, an adenovirus vector, a lentivirus vector, a herpesvirus vector or a retroviral vector. In one embodiment, a cell comprises the vector. In one embodiment, the cell is a mammalian cell or plant cell. In one embodiment, the cell is a mammalian hematopoietic stem cell. Further provided is isolated polypeptide encoded by the vector, e.g., a polypeptide comprising a modified hexosaminidase beta-subunit that forms homodimers, a linker and an ApoE peptide that allows for transport across the blood brain barrier.

Also provided is a method to prevent, inhibit or treat one or more symptoms of GM2-gangliosidosis in a mammal. The method includes administering to the mammal an effective amount of a composition comprising the vector, the cell having the vector, or the polypeptide encoded by the vector. In one embodiment, the mammal is a human. In one embodiment, the human has Sandhoff disease or Tay-Sachs disease. In one embodiment, the composition is systemically administered. In one embodiment, the composition is injected. In one embodiment, the composition is administered to the central nervous system. In one embodiment, the composition is administered to the cerebrospinal fluid. In one embodiment, the composition comprises particles or liposomes comprising the vector. In one embodiment, the vector comprises RNA. In one embodiment, the particles are nanoparticles.

In one embodiment, the disclosure provides for delivery of one or more genes encoding proteins for a gene editing system, e.g., CRISPR/Cas, TALENs, zinc finger nuclease or homing endonucleases, delivered via one or more vectors such as plasmids or viral vectors, including but not limited to lentivirus vectors, adenovirus vectors, adeno-associated virus (AAV) vectors, e.g., AAV2, AAV5, AAV6, AAV8, or AAV9, or herpesvirus vectors, which proteins may be useful to prevent, inhibit or treat diseases such as monogenic diseases. In one embodiment, at least one or two vectors are used to deliver one or more CRISPR components, e.g., nucleic acid encoding Cas, gRNA(s), a gene encoding the protein or interest, e.g., which is optionally promoterless, for targeted insertion into the genome of a human cell, e.g., ex vivo or in vivo. In one embodiment, systemic of the one or more vectors administration is employed. In one embodiment, Cas may be supplied in trans. Combinations of different vectors and/or proteins may be used. Sequences for gRNA and homology arms flanking the gene of interest may be directed to any insertion (target) site in the genome of a human cell so long as the site allows for adequate expression of the introduced gene. Exemplary insertion sites include but are not limited to the albumin locus, AAVS1, Rosa26, CCR5, HPRT, or the alpha fetoprotein locus, e.g., intron 1 of the human albumin locus, AAVS1, Rosa26, CCR5, HPRT, or the alpha fetoprotein locus. In one embodiment, a human genome site (a locus) for insertion of a gene of interest has few if any polymorphisms, e.g., selected gRNA(s) and/or homology arm sequences are useful for more than one individual as the sequences at and near the insertion site are conserved among genetically unrelated individuals. In one embodiment, the gRNA sequence is directed to a conserved sequence. In one embodiment, where the locus is polymorphic, the gRNA sequence may be directed to a conserved sequence and the homology arms may have a polymorphic sequence, e.g., the homology arms are specific for an individual. In one embodiment, where the locus is polymorphic, the gRNA sequence and the homology arms may have polymorphic sequences, e.g., both the gRNA and the homology arms are specific for an individual. In one embodiment, the vector(s) is/are mRNA, e.g., in a nanoparticle such as a liposome. In one embodiment, the vector(s) is/are plasmid vectors, e.g., in a nanoparticle such as a liposome. In one embodiment, the vector(s) is/are viral vectors. In one embodiment, the viral vectors is an adeno-associated virus vector. In one embodiment, one vector is employed. In one embodiment, two vectors are employed.

In one embodiment, a method to prevent, inhibit or treat a GM2-gangliosidosis disease in a mammal or a mammalian cell is provided. In one embodiment, the method includes administering an effective amount of i) Cas or an isolated nucleic encoding Cas, e.g., a vector comprising an isolated nucleic encoding Cas, and ii) isolated nucleic acid for one or more gRNAs comprising a targeting sequence for a genomic target and nucleic acid comprising a coding sequence for a prophylactic or therapeutic gene product flanked by homology arms, e.g., a vector comprising isolated nucleic acid for one or more gRNAs comprising a targeting sequence for a genomic target and nucleic acid comprising a coding sequence for a prophylactic or therapeutic gene product flanked by homology arms, or an effective amount of iii) isolated nucleic encoding Cas and nucleic acid for one or more gRNAs comprising a targeting sequence for a genomic target, e.g., a vector comprising isolated nucleic encoding Cas and nucleic acid for one or more gRNAs comprising a targeting sequence for a genomic target, and iv) isolated nucleic acid comprising a coding sequence for a prophylactic or therapeutic gene product flanked by homology arms, e.g., a vector comprising isolated nucleic acid comprising a coding sequence for a prophylactic or therapeutic gene product flanked by homology arms, wherein the expression of the coding sequence in the mammal prevents, inhibits or treats the disease or in the mammalian cell results in increased expression of the prophylactic or therapeutic gene product. In one embodiment, the mammal is a human. In one embodiment, at least one homology arm has one or more mutations that decrease subsequent cleavage events by the introduced recombinase, e.g., Cas9. In one embodiment, a composition comprises Cas9 or an isolated nucleic encoding Cas9, and isolated nucleic acid for one or more gRNAs comprising a targeting sequence for a genomic target and nucleic acid comprising a coding sequence for a prophylactic or therapeutic gene product flanked by homology arm. In one embodiment, the Cas is SpCas9. In one embodiment, the Cas is SaCas9. In one embodiment, a composition comprises isolated nucleic encoding Cas9 and nucleic acid for one or more gRNAs comprising a targeting sequence for a genomic target, and isolated nucleic acid comprising a coding sequence for a prophylactic or therapeutic gene product flanked by homology arms. In one embodiment, the targeting sequence targets intron 1 of the albumin locus. In one embodiment, the targeting sequence comprises at least 20 contiguous nucleotides in intron 1 of the albumin locus.

### BRIEF DESCRIPTION OF THE FIGURES

FIG. 1. IDUA activity in treated MPS I and control mice. Mean±standard errors. ** p<0.01, **** p<0.0001.

FIG. 2. MUGS and MUG activity in the liver of treated Sandhoff and control mice. Mean±standard errors. **** p<0.0001.

FIG. 3. PS Gene Editing to insert therapeutic transgene into the albumin locus.

FIG. 4. Liver is a target for treating genetic disorders.

FIG. 5. Integration at the albumin locus leads to high transgene expression.

FIG. 6. Exemplary study design and overall strategy.

FIG. 7. GM2-gangliosidoses.

FIG. 8. Potential cDNA donors.

FIG. 9. HEXX and HEXO.

FIG. 10. Selection of a HEXO candidate.

FIG. 11. HEXO achieved significantly improved survival rate than HEXX and HEXM.

FIG. 12. Plasma β-hexosaminidase enzyme activities increased significantly.

FIG. 13. Rotarod and mesh test showed improved coordination, motor function, and grip strength in treated mice.

FIG. 14. Open field test showed improved motor function and habituation in treated mice.

FIG. 15. Pole test showed reduced bradykinesia in treated mice.

FIG. 16. Cellular vacuolation was markedly reduced in the liver of treated Sandhoff mice.

FIG. 17. Cellular vacuolation was markedly reduced in the brain of treated Sandhoff mice.

FIG. 18. Low antibody levels against AAV8 and Cas9 in treated mice.

FIG. 19. Cytokine markers were not affected.

FIG. 20. Plasma AST, ALT, and creatinine levels were not affected.

FIG. 21. Summary.

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The terms “nucleic acid,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

An “isolated” polynucleotide, e.g., plasmid, virus, polypeptide, cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. In one embodiment, a recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The invention also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s)are outweighed by the therapeutically beneficial effects.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are eleven serotypes of AAVs, AAV1-AAV11, including AAV2, AAV5, AAV6, AAV8, AAV9 and AAVrh10, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector may be homologous, i.e., from the same serotype, heterologous, i.e., from different serotypes, or chimeric, i.e., an ITR which has ITR sequences from more than one AAV serotype.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), e.g., between about 100 and 1,000 nucleotides in length (or any integer therebetween), e.g., between about 200 and 500 nucleotides in length.

For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

### Gene Delivery Vectors

Gene delivery vectors include, for example, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med. 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

Exemplary rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

### Exemplary Compositions and Routes of Delivery for the Compositions

Any route of administration may be employed so long as that route and the amount administered are prophylactically or therapeutically useful.

In vivo administration of the components, e.g., delivered in a viral vector such as a lentivirus or AAV vector, and compositions containing them, isolated recombinant cells, isolated polypeptides, or delivery systems such as nanoparticles or liposomes having the vector or polypeptide, can be accomplished by any suitable method and technique presently or prospectively known to those skilled in the art. The subject polynucleotides or polypeptides can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intracisternal administration, such as by injection.

Administration of the compositions can be a single administration, or at continuous or distinct intervals as can be readily determined by a person skilled in the art. In one embodiment, a polynucleotide component is stably incorporated into the genome of a person of animal in need of treatment. Methods for providing gene therapy are well known in the art.

The compositions can also be administered utilizing liposome and nano-technology, slow release capsules, implantable pumps, and biodegradable containers, and orally or intestinally administered intact plant cells expressing the therapeutic product. These delivery methods can, advantageously, provide a uniform dosage over an extended period of time.

Suitable dose ranges for are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or10¹⁷ viral genomes or infectious units of viral vector delivered in about 10, 50, 100, 200, 500, 1000, or 2000 microliters. In one embodiment, the amount of a vector to be administered, e.g., to a human, is about 1 ng to 1 g, e.g., about 1 ng to 100 ng, 100 ng to 250 ng, 250 ng to 750 ng, 750 ng to 1000 ng, 1 mg to 100 mg, 100 mg to 250 mg, 250 mg to 750 mg, or 750 mg to 1000 mg. In one embodiment, the amount of recombinant cells expressing the polypeptides disclosed herein to be administered, e.g., to a human, is about 10⁶ to 10¹⁵ cells, e.g., about 10⁶ to 10⁷, 10⁷ to 10⁸, 10⁸ to 10⁹, 10⁹ to 10¹⁰, 10¹⁰ to 10¹¹, 10¹¹ to 10¹², 10¹² to 10¹³, 10¹³ to 10¹⁴, or 10¹⁴ to 10¹⁵ cells. In one embodiment, the amount of virus to be administered, e.g., to a human, is about 10⁶ to 10¹⁵ viral genomes, focus forming units (FFU) or infectious units (IU), e.g., about 10⁶ to 10⁷, 10⁷ to 10⁸, 10⁸ to 10⁹, 10⁹ to 10¹⁰, 10¹⁰ to 10¹¹, 10¹¹ to 10¹², 10¹² to 10¹³, 10¹³ to 10¹⁴, or 10¹⁴ to 10¹⁵, viral genomes, FFU or IU. In one embodiment, the amount of a protein to be administered, e.g., to a human, is about 1 mg to 1 g, e.g., 1 mg to 100 mg, 100 mg to 250 mg, 250 mg to 750 mg, or 750 mg to 1000 mg. It should be understood that the aforementioned dosage is merely an exemplary dosage and those of skill in the art will understand that this dosage may be varied. Effective doses may be extrapolated from dose-responsive curves derived from in vitro or in vivo test systems.

In one embodiment, suitable dose ranges are generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters, of single injection volume. For instance, viral genomes or infectious units of vector per microliter would generally contain about 10⁴, 10⁶, 10⁶, 10⁷, 10⁸, 10⁹, 10¹³, 10¹¹, 10¹², 10¹³, or 10¹⁴ viral genomes or infectious units (IU) of viral vector. In one embodiment, suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁶, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units.

Administration of vectors, polypeptides or cells in accordance with the present invention can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment, the vector, polypeptide or cells may be administered by any route including parenterally. In one embodiment, the vector, polypeptide or cells may be administered by subcutaneous, intramuscular, or intravenous injection, orally, intrathecally, or intracranially, or by sustained release, e.g., using a subcutaneous implant. The agent(s) may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, the agent(s) may comprise an aqueous solution of a water soluble pharmaceutically acceptable salt of the active acids according to the invention, desirably in a concentration of 0.01-10%, and optionally also a stabilizing agent and/or buffer substances in aqueous solution. Dosage units of the solution may advantageously be enclosed in ampules.

The vector, polypeptide or cells may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the vector, polypeptide or cells is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the vector, polypeptide or cells is administered may vary within a wide range and will depend on various factors such as the severity of the disease, the age of the patient, etc., and may have to be individually adjusted. Compositions described herein may be employed in combination with another medicament. The compositions can appear in conventional forms, for example, aerosols, solutions, suspensions, or topical applications, or in lyophilized form.

Typical compositions include the vector, polypeptide or cells and a pharmaceutically acceptable excipient which can be a carrier or a diluent. For example, the vector, polypeptide or cells may be mixed with a carrier, or diluted by a carrier, or enclosed within a carrier. When the vector, polypeptide or cells is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the vector, polypeptide or cells. Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The vector, polypeptide or cells may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present invention may include, for example, micelles or liposomes, or some other encapsulated form, such as nanoparticles, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few μm.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swallen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition of the invention can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

### Exemplary Compositions and Methods

In one embodiment, nucleic acid encoding a modified form of HexB that is linked to a targeting peptide, thereby providing a fusion polypeptide, is provided. The modified form of HexB (Mod2B) has substitutions relative to the unmodified form of HexB. Mod2B forms homodimers. The nucleotide sequence encoding Mod2B was modified by replacing codons to increase expression thereof (yielding HEXX) and that sequence was fused to a targeting peptide via a linker (yielding, in one embodiment HEXO). The resulting vector may be administered as, for example, DNA, e.g., a plasmid, or as nucleic acid encapsulated in particles or complexed with molecules, introduced to a virus vector such as an adeno-associated viral vector, introduced to cells such as CHO cells, which cells are useful to express Mod2B linked to a targeting peptide, or introduced to hematopoietic cells, e.g., via a viral vector. In one embodiment, the vector may be based on a persistent expression vector such as an adeno-associated virus (AAV) vector (but could be another viral vector such as a retro- or lenti-virus vector).

Any linker may be employed so long as it does not inhibit the activity of the hexosaminidase and/or the targeting peptide, e.g., relative to the activity of a hexosaminidase without a linker and without a targeting peptide, relative to the activity of a hexosaminidase without a linker and with a targeting peptide or relative to the activity of a hexosaminidase with a linker and without a targeting peptide. Exemplary linkers include but are not limited to (GGGGS)₃ (SEQ ID NO:20), (GGGGS)₂ (SEQ ID NO:21), (GGGGS)₄ (SEQ ID NO:22), (Gly)₈, (Gly)₆, (EAAAK)₃ (SEQ ID NO:23), (EAAAK)ₙ (SEQ ID NO:24) (n=1-3), A(EAAAK)₄(ALEA(EAAAK)₄A (SEQ ID NO:25), A(EAAAK)₃(ALEA(EAAAK)₃A (SEQ ID NO:26), A(EAAAK)₂(ALEA(EAAAK)₂A (SEQ ID NO:27), A(EAAAK)₅(ALEA(EAAAK)₆A (SEQ ID NO:28), GGGGS (SEQ ID NO:12), PAPAP (SEQ ID NO:30), AEAAAKEAAAKA (SEQ ID NO:31), (GGGGS)ₙ (SEQ ID NO:32) (n=1, 2, 4), (Ala-Pro)ₙ (10-34 aa), X₁DX₂X₃E (SEQ ID NO:42), wherein X₁, X₂ or X₃ individually is I, L, V, A or G, X₁X₄X₂X₃X₅ (SEQ ID NO:48), wherein X₄ or X₅ individually is D, E, R, K, N or Q, IDLE (SEQ ID NO:43), X₆X₇X₈X₉X₁₀ (SEQ ID NO:44), wherein each of X₆, X₇, X₈ and X₉ individually is I, L, V, A or G and wherein X₁₀ is S or T or X₆X₇X₁₀, X₆X₁₀X₁₀, GGGGS (SEQ ID NO:12), GGS, GSS, GSSSSSS (SEQ ID NO:13) or LGGGGSGGGGSGGGGSGGGGS (SEQ ID NO:47). or disulfide, and optionally includes a protease cleavage site such as VSQTSKLTR ↓AETVFPDV^{b} (SEQ ID NO:33), PLG ↓LWA^{c} (SEQ ID NO:34), RVL ↓AEA (SEQ ID NO:35); EDVVCC ↓SMSY (SEQ ID NO:36); GGIEGR ↓GS^{c} (SEQ ID NO:37) TRHRQPR ↓GWE (SEQ ID NO:38); AGNRVRR ↓SVG (SEQ ID NO:39); RRRRRRR ↓R↓^{d} GFLG ↓^{e} (SEQ ID NO:40).

In one embodiment, the targeting peptide comprises X₁₁X₁₂X₁₃X₁₄X₁₆X₁₇X₁₈X₁₉ (SEQ ID NO:49), wherein each of X₁₁, X₁₄, X₁₅, X₁₈, and X₁₉ individually is I, L, V, A or G, and wherein each of X₁₂, X₁₃, X₁₅ X₁₆, and X₁₇ is R, K or H, e.g., LRKLRKRLL (SEQ ID NO:50), or multimers thereof.

In one embodiment, the hexosaminidase nucleotide sequence has at least 80%, 85%, 88%, 90%, 92%, 94%, 95%, 96%, 97%, 98%, 99% or more nucleic acid sequence identity to SEQ ID NO:2 but in one embodiment does not have SEQ ID NO:1. In one embodiment, the encoded hexosaminidase has at least 80%, 85%, 88%, 90%, 92%, 94%, 95%, 96%, 97%, 98%, 99% or more amino acid sequence identity to a polypeptide encoded by SEQ ID NO:2. In one embodiment, the encoded hexosaminidase does not have an amino acid sequence encoded by SEQ ID NO:1. In one embodiment, the encoded hexosaminidase has about 2%, 5%, 10%, 12%, 15% or up to 20% fewer residues than a hexosaminidase encoded by SEQ ID NO:2.

In one embodiment, the composition comprises, consists essentially of, or consists of the above-described vector(s) or recombinant viruses having vector sequences and a pharmaceutically acceptable (e.g., physiologically acceptable) carrier. When the composition consists essentially of the vector and a pharmaceutically acceptable carrier, additional components can be included that do not materially affect the composition (e.g., adjuvants, buffers, stabilizers, anti-inflammatory agents, solubilizers, preservatives, etc.). When the composition consists of vector and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the inventive gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. The therapeutically effective amount may be between 1×10¹⁰ genome copies to 1×10¹³ genome copies.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence as described above.

### Routes of Administration, Dosages and Dosage Forms

Administration of the gene delivery vectors in accordance with the present invention may be continuous or intermittent, depending, for example, upon the recipient's physiological condition, and other factors known to skilled practitioners. The administration of the gene delivery vector(s) may be essentially continuous over a preselected period of time or may be in a series of spaced doses. Both local administration, e.g., intracranial, intranasal or intrathecal, and systemic administration, e.g., using viruses that cross the blood-brain barrier, are contemplated. Any route of administration may be employed, e.g., intravenous, intranasal or intrabronchial, direct administration to the lung and intrapleural. In one embodiment, compositions may be delivered to the pleura.

One or more suitable unit dosage forms comprising the gene delivery vector(s), which may optionally be formulated for sustained release, can be administered by a variety of routes including intracranial, intrathecal, or intranasal, or other means to deliver to the CNS, or oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s) administered to achieve a particular outcome will vary depending on various factors including, but not limited to, the genes and promoters chosen, the condition, patient specific parameters, e.g., height, weight and age, and whether prevention or treatment, is to be achieved.

Vectors of the invention may conveniently be provided in the form of formulations suitable for administration, e.g., into the brain. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician, but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, administration may be by intracranial, intrahepatic, intratracheal or intrabronchial injection or infusion using an appropriate catheter or needle. A variety of catheters may be used to achieve delivery, as is known in the art. For example, a variety of general purpose catheters, as well as modified catheters, suitable for use in the present invention are available from commercial suppliers. Also, where delivery is achieved by injection directly into a specific region of the brain or lung, a number of approaches can be used to introduce a catheter into that region, as is known in the art.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the gene delivery vectors can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions appropriate for parenteral administration, for instance, by intramuscular, subcutaneous or intravenous routes.

The pharmaceutical formulations of the vectors can also take the form of an aqueous or anhydrous solution, e.g., a lyophilized formulation, or dispersion, or alternatively the form of an emulsion or suspension.

In one embodiment, the vectors may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease, e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Gas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRN

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula. Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987) Proc. Natl. Acad. Sci. USA 84:4959-4963; Nehls, et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Exemplary Embodiments

The disclosure provides for a nucleic acid vector encoding a fusion polypeptide comprising a modified hexosaminidase beta-subunit that forms homodimers, a linker and an ApoE peptide that allows for transport across the blood brain barrier. In one embodiment, the vector is a plasmid. In one embodiment, the vector is a viral vector. In one embodiment, the vector encodes a multimer of the ApoE peptide. In one embodiment, the hexoseaminidase beta-subunit is encoded by SEQ ID NO:1, SEQ ID NO:2 or SEQ ID NO:3 or has at least 90% amino acid sequence identity to the hexoseaminidase beta-subunit is encoded by SEQ ID NO:1, SEQ ID NO:2 or SEQ ID NO:3 and includes one or more of G, S, En or P at residue 312, 313, 314 or 315, respectively, S, G or T at residue 316, 317 or 318, respectively, or N or R at residue 452 or 453, respectively, or any combination thereof. In one embodiment, the vector comprises a nucleotide sequence having at least 80%, 90% or 95% nucleic acid sequence identity to SEQ ID NO:2 or SEQ ID NO:3. In one embodiment, the linker comprises X₁DX₂X₃E, wherein X₁, X₂ or X₃ individually is I, L, V, A or G. In one embodiment, the linker comprises X₁X₄X₂X₃X₅, wherein X₄ or X₅ individually is D, E, R, K, N or Q. In one embodiment, the linker comprises IDILE. In one embodiment, the linker comprises X₆X₇X₈X₉X₁₀, wherein each of X₆, X₇, X₈ and X₉ individually is I, L, V, A or G and wherein X₁₀ is S or T or X₆X₇X₁₀, X₆X₁₀X₁₀. In one embodiment, the linker comprises GGGGS, GGS, GSS, or GSSSSSS. In one embodiment, the linker comprises LGGGGSGGGGSGGGGSGGGGS. In one embodiment, the ApoE peptide comprises X₁₁X₁₂X₁₃X₁₄X₁₅X₁₆X₁₇X₁₈ X₁₉, wherein each of X₁₁, X₁₄, X₁₅, X₁₈, and X is individually is I, L, V, A or G, and wherein each of X₁₂, X₁₃, X₁₅ X₁₆, and X₁₇ is R, K or H. In one embodiment, the ApoE peptide comprises LRKLRKRLL. In one embodiment, the vector is an adeno-associated virus vector, an adenovirus vector, a lentivirus vector, a herpesvirus vector or a retroviral vector. In one embodiment, the vector comprises an open reading frame for the fusion polypeptide which operably links an open reading frame for the modified hexosaminidase beta-subunit that forms homodimers via the nucleotide sequence encoding the linker to the open reading frame for the ApoE peptide. In one embodiment, the modified hexosaminidase beta-subunit in N-terminal to the ApoE peptide. In one embodiment, the modified hexosaminidase beta-subunit in C-terminal to the ApoE peptide.

Also provided is an isolated cell comprising the vector. In one embodiment, the cell is a mammalian cell or plant cell. In one embodiment, the cell is a mammalian hematopoietic stem cell.

Further provided is a polypeptide encoded by the vector of or expressed by the cell, such as a polypeptide comprising a modified hexosaminidase beta-subunit that forms homodimers, a linker and an ApoE peptide that allows for transport across the blood brain barrier.

The disclosure provides a pharmaceutical composition comprising the vector or isolated polypeptide.

Also provided is a method to prevent, inhibit or treat one or more symptoms of GM2-gangliosidosis in a mammal, comprising: administering to the mammal an effective amount of a composition comprising the vector, the cell, or the polypeptide. In one embodiment, the mammal is a human. In one embodiment, the human has Sandhoff disease or Tay-Sachs disease. In one embodiment, the composition is systemically administered. In one embodiment, the composition is injected. In one embodiment, the composition is administered to the central nervous system. In one embodiment, the composition is administered to the cerebrospinal fluid. In one embodiment, the composition comprises particles or liposomes comprising the vector. In one embodiment, the vector comprises RNA. In one embodiment, the particles are nanoparticles. In one embodiment, the administration provides for an extended lifespan, e.g., relative to corresponding mammals administered a vector encoding, or a polypeptide comprising HEXM or HEXX.

The invention will be further described by the following non-limiting examples.

### Example I

Two sequences were tested to assess the efficacy of ApoE in facilitating lysosomal enzyme α-L-iduronidase (IDUA) cross the blood brain barrier.
IDUAF1: IDUA+molecular cloning site (RSSATRVD; SEQ ID NO:51)+c-myc tag (EQKLISEEDL; SEQ ID NO:52+linker (IDILE; SEQ ID NO:6)+repeat of aa 159-167 of ApoE (LRKLRKRLLLRKLRKRLL; SEQ ID NO:7)
IDUAF2: IDUA+linker (LGGGGSGGGGSGGGGSGGGGS; SEQ ID NO:8)+repeat of aa 141-149 of ApoE (LRKLRKRLLLRKLRKRLL; SEQ ID NO:7)

Hydrodynamic injections of plasmids encoding IDUAF1 and IDUAF2 in comparison with native IDUA sequence were assessed in a murine model of MPS I disease. These plasmids were exactly the same except for the cDNA sequence (IDUAF1, IDUF2, or IDUA). To limit the transgene expression in the liver, the cDNA sequence was under the control of a liver-specific human al-antitrypsin (hAAT) promoter. After 48 hours, the mice were transcardially perfused with 35 mL phosphate buffered saline (PBS), and tissues were harvested.

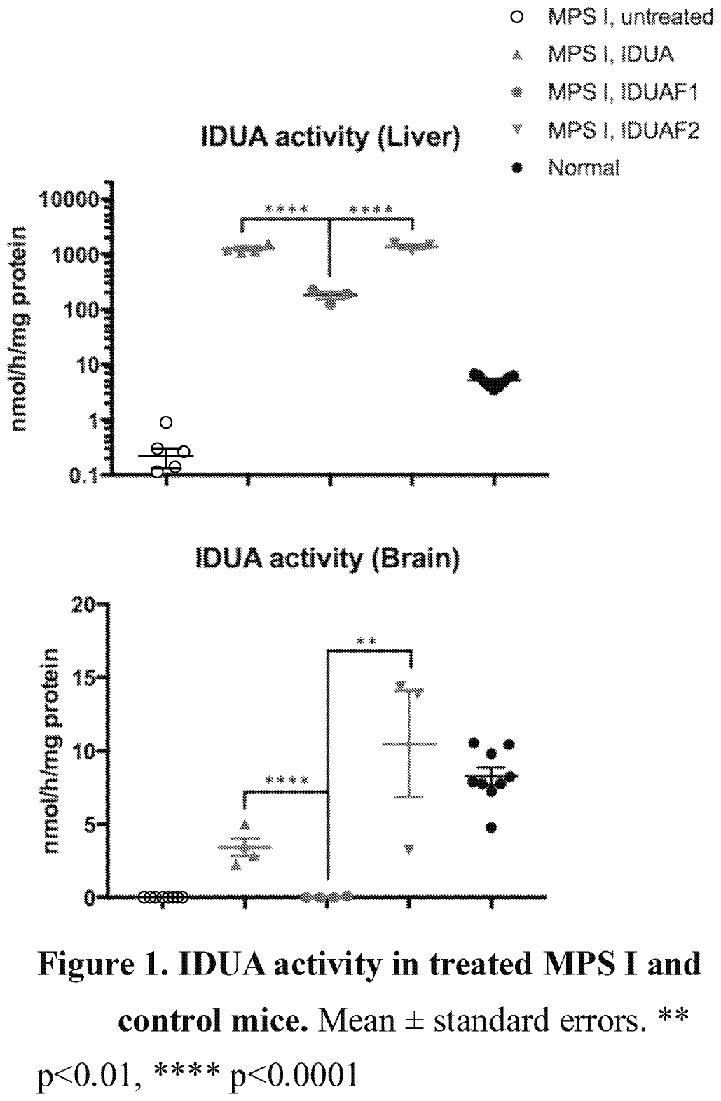

As shown in FIG. 1, IDUAF1 achieved significantly lower enzyme activity in the liver than the native IDUA sequence (179±29 vs 1,253±115 nmol/h/mg), indicating that such a linker and ApoE sequence had a negative impact on the stability or catalytic activity. In contrast, IDUAF2 achieved high enzyme activity in the liver (1,364±125 nmol/h/mg), indicating that this specific linker and ApoE sequence did not affect stability and catalytic activity.

IDUA achieved significant enzyme activity in the brain. Since the transgene expression was limited in the liver by a liver-specific promoter and perfusion was performed, the increased enzyme activity in the brain should not result from transgene expression from the brain or blood contamination. Previous studies have shown that a high and constant level of lysosomal enzyme in the blood can facilitate a small amount enter the brain (Dunder et al., 2000; Roces et al., 2004; Lee et al., 2005; Matzner et al., 2005; Volger et al., 2007; Blanz et al., 2008; Matzner et al., 2009; Polito et al., 2010; Rozaklis et al., 2011; Ou et al.,2014). Therefore, the enzyme activity expressed and secreted from the liver entered the brain through blood circulation. More interestingly, IDUAF2 achieved significantly higher enzyme activity in the brain than the native IDUA sequence (10.5±3.6 vs 3.4±0.6 nmol/h/mg). Therefore, IDUAF2 showed higher efficiency in entering the brain than the native IDUA. In contrast, IDUAF1 did not achieve above background enzyme activity in the brain. These results demonstrated that IDUAF2 is a novel construct that maintains comparable enzyme activity to native IDUA and has higher efficiency in entering the brain.

Based on these results, repeats of amino acid 141-149 of ApoE (LRKLRKRLLLRKLRKRLL) (SEQ ID NO:7) and the linker sequence (LGGGGSGGGGSGGGGSGGGGS) (SEQ ID NO:8) were added to the C terminus of the protein encoded by HEXX. The resulting fusion construct was designated as HEXO. Then, hydrodynamic injections of plasmids encoding HEXX and HEXO in comparison with HEXM in a murine model of Sandhoff disease. These plasmids were exactly the same except for the cDNA sequence (HEXX, HEXO or HEXM). To limit transgene expression in the liver, the cDNA sequence was under the control of a liver-specific human al-antitrypsin (hAAT) promoter. After 48 hours, the mice were transcardially perfused with 35 mL phosphate buffered saline (PBS), and tissues were harvested. Enzyme assays with two different man-made substrates, 4-Methylumbelliferyl N-acetyl-b-D-glucosaminide (MUG) and 4-Methylumbelliferyl-6-sulfa-2-Acetoamido-2-Deoxy-beta-D-Glucopyranoside Potassium salt (MUGS), were performed. MUG activity reflects the catalytic activity of all Hex isozymes (S, A, and B), while MUGS activity reflects catalytic activity of Hex isozyme A.

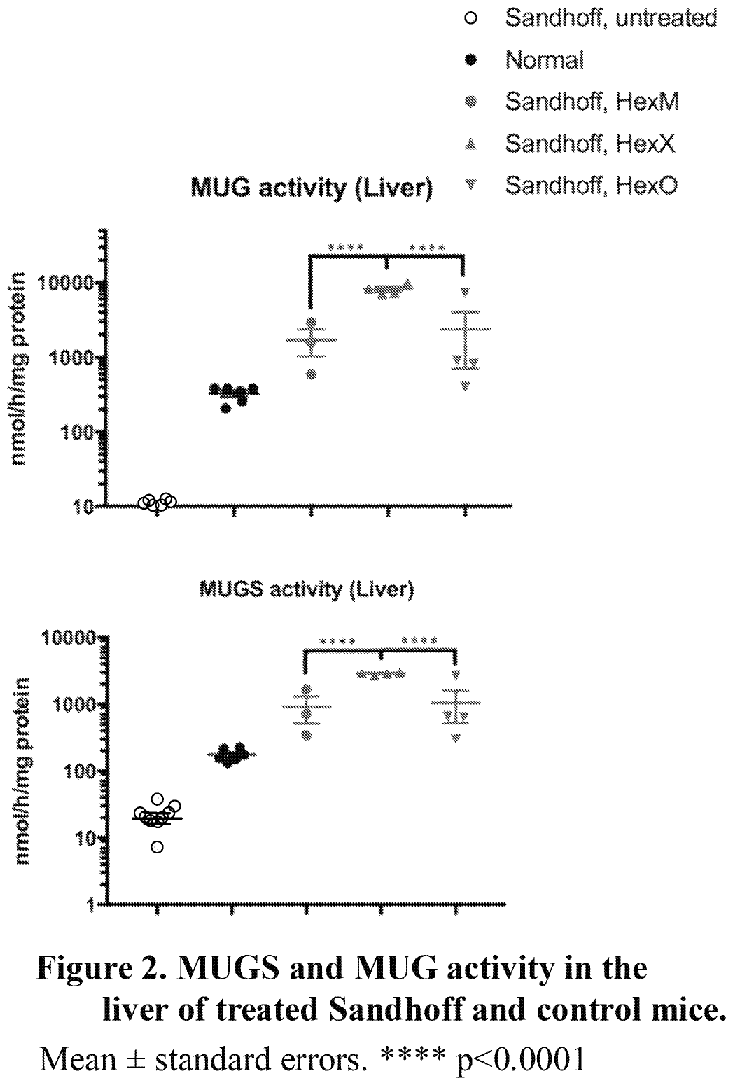

As shown in FIG. 2, all three constructs (HEXX, HEXO, and HEXM) achieved significant MUGS and MUG activities, indicating that HEXX and HEXO can also form homodimers as HEXM. HEXX achieved significantly higher MUG activity (8,230±673 nmol/h/mg) than HEXO (2,355±1651 nmol/h/mg) and HEXM (1,702±671 nmol/h/mg). Moreover, HEXX achieved significantly higher MUGS activity (2,901±72 nmol/h/mg) than HEXO (1,849±534 nmol/h/mg) and HEXM (1,052±395 nmol/h/mg). These results demonstrated that HEXX is superior to HEXM for its higher transgene expression level, and HEXO is superior to HEXM and Mod2B for its potential in entering the brain.

(SEQ ID NO: 1)
Mod2B
ATGGAGCTGTGCGGGCTGGGGCTGCCCCGGCCGCCCATGC
TGCTGGCGCTGCTGTTGGCGACACTGCTGGCGGCGATGTT
GGCGCTGCTGACTCAGGTGGCGCTGGTGGTGCAGGTGGCG
GAGGCGGCTCGGGCCCCGAGCGTCTCGGCCAAGCCGGGGC
CGGCGCTGTGGCCCCTGCCGCTCTTGGTGAAGATGACCCC
GAACCTGCTGCATCTCGCCCCGGAGAACTTCTACATCAGC
CACAGCCCCAATTCCACGGCGGGCCCCTCCTGCACCCTGC
TGGAGGAAGCGTTTCGACGATATCATGGCTATATTTTTGG
TTTCTACAAGTGGCATCATGAACCTGCTGAATTCCAGGCT
AAAACCCAGGTTCAGCAACTTCTTGTCTCAATCACCCTTC
AGTCAGAGTGTGATGCTTTCCCCAACATATCTTCAGATGA
GTCTTATACTTTACTTGTGAAAGAACCAGTGGCTGTCCTT
AAGGCCAACAGAGTTTGGGGAGCATTACGAGGTTTAGAGA
CCTTTAGCCAGTTAGTTTATCAAGATTCTTATGGAACTTT
CACCATCAATGAATCCACCATTATTGATTCTCCAAGGTTT
TCTCACAGAGGAATTTTGATTGATACATCCAGACATTATC
TGCCAGTTAAGATTATTCTTAAAACTCTGGATGCCATGGC
TTTTAATAAGTTTAATGTTCTTCACTGGCACATAGTTGAT
GACCAGTCTTTCCCATATCAGAGCATCACTTTTCCTGAGT
TAAGCAATAAAGGAAGCTATTCTTTGTCTCATGTTTATAC
ACCAAATGATGTCCGTATGGTGATTGAATATGCCAGATTA
CGAGGAATTCGAGTCCTGCCAGAATTTGATACCCCTGGGC
ATACACTATCTTGGGGAAAAGGTCAGAAAGACCTCCTGAC
TCCATGTTACAGTGGGTCTGAGCCCTCTGGCACCTTTGGA
CCTATAAACCCTACTCTGAATACAACATACAGCTTCCTTA
CTACATTTTTCAAAGAAATTAGTGAGGTGTTTCCAGATCA
ATTCATTCATTTGGGAGGAGATGAAGTGGAATTTAAATGT
TGGGAATCAAATCCAAAAATTCAAGATTTCATGAGGCAAA
AAGGCTTTGGCACAGATTTTAAGAAACTAGAATCTTTCTA
CATTCAAAAGGTTTTGGATATTATTGCAACCATAAACAAG
GGATCCATTGTCTGGCAGGAGGTTTTTGATGATAAAGCAA
AGCTTGCGCCGGGCACAATAGTTGAAGTATGGAAAGACAG
CGCATATCCTGAGGAACTCAGTAGAGTCACAGCATCTGGC
TTCCCTGTAATCCTTTCTGCTCCTTGGTACTTAAACCGTA
TTAGCTATGGACAAGATTGGAGGAAATACTATAAAGTGGA
ACCTCTTGATTTTGGCGGTACTCAGAAACAGAAACAACTT
TTCATTGGTGGAGAAGCTTGTCTATGGGGAGAATATGTGG
ATGCAACTAACCTCACTCCAAGATTATGGCCTCGGGCAAG
TGCTGTTGGTGAGAGACTCTGGAGTTCCAAAGATGTCAGA
GATATGGATGACGCCTATGACAGACTGACAAGGCACCGCT
GCAGGATGGTCGAACGTGGAATAGCTGCACAACCTCTTTA
TGCTGGATATTGTAACCATGAGAACATGTAA.

An exemplary amino acid sequence for Mod2B is:

(SEQ ID NO: 24)
MELCGLGLPRPPMLLALLLATLLAAMLALLTQVALVVQVA
EAARAPSVSAKPGPALWPLPLLVKMTPNLLHLAPENFYIS
HSPNSTAGPSCTLLEEAFRRYHGYIFGFYKWHHEPAEFQA
KTQVQQLLVSITLQSECDAFPNISSDESYTLLVKEPVAVL
KANRVWGALRGLETFSQLVYQDSYGTFTINESTIIDSPRF
SHRGILIDTSRHYLPVKIILKTLDAMAFNKFNVLHWHIVD
DQSFPYQSITFPELSNKGSYSLSHVYTPNDVRMVIEYARL
RGIRVLPEFDTPGHTLSWGKGQKDLLTPCYSGSEPSGTFG
PINPTLNTTYSFLTTFFKEISEVFPDQFIHLGGDEVEFKC
WESNPKIQDFMRQKGFGTDFKKLESFYIQKVLDIIATINK
GSIVWQEVFDDKAKLAPGTIVEVWKDSAYPEELSRVTASG
FPVILSAPWYLNRISYGQDWRKYYKVEPLDFGGTQKQKQL
FIGGEACLWGEYVDATNLTPRLWPRASAVGERLWSSKDVR
DMDDAYDRLTRHRCRMVERGIAAQPLYAGYCNHENM
HEXX
(SEQ ID NO: 2)
ATGGAGCTTTGTGGACTTGGCCTTCCACGACCACCCATGC
TCTTGGCTTTGTTGCTGGCCACCCTTTTGGCAGCCATGCT
TGCCCTTCTCACACAGGTAGCACTCGTCGTACAGGTCGCT
GAGGCAGCAAGGGCTCCCAGCGTATCAGCAAAGCCCGGCC
CAGCACTCTGGCCATTGCCCCTTCTGGTGAAGATGACTCC
AAACCTGCTGCATTTGGCCCCCGAGAACTTCTACATCTCT
CATAGCCCTAACAGCACCGCCGGCCCTTCATGTACTCTCC
TTGAAGAAGCATTCAGACGATATCATGGGTATATCTTTGG
ATTCTATAAGTGGCACCATGAGCCAGCAGAGTTCCAGGCC
AAAACACAGGTGCAGCAACTGCTCGTATCCATTACTCTGC
AGTCTGAATGCGACGCATTCCCAAATATTTCATCTGACGA
GTCCTACACTCTTCTTGTAAAAGAGCCCGTTGCTGTCCTG
AAAGCAAATAGGGTTTGGGGAGCCTTGAGAGGATTGGAGA
CCTTCTCACAACTCGTTTATCAAGATTCCTATGGAACTTT
TACCATCAACGAGTCAACCATAATTGATTCCCCCAGATTC
TCACACCGGGGCATTCTGATAGATACATCCCGCCACTATC
TCCCCGTAAAAATTATACTTAAAACCCTTGATGCAATGGC
TTTCAACAAATTTAACGTGTTGCATTGGCACATCGTCGAC
GACCAGAGTTTTCCCTATCAAAGTATCACATTCCCCGAAC
TCTCTAATAAGGGTAGCTACAGTTTGTCCCATGTATACAC
ACCCAACGATGTACGGATGGTTATAGAGTATGCCAGGCTG
CGGGGTATACGAGTCCTTCCAGAgTTCGACACACCTGGCC
ACACATTGAGTTGGGGCAAGGGACAAAAGGACCTTCTTAC
ACCATGCTACAGCGGCTCTGAGCCCAGTGGGACATTCGGT
CCCATTAACCCTACACTCAACACTACCTACAGTTTTCTTA
CCACTTTTTTTAAGGAAATTTCTGAAGTCTTCCCTGACCA
ATTCATACATCTTGGCGGAGACGAAGTAGAGTTCAAATGC
TGGGAATCAAACCCTAAGATCCAGGACTTTATGAGACAAA
AAGGATTTGGCACCGATTTCAAAAAGCTGGAGTCCTTTTA
CATTCAGAAAGTTTTGGATATTATCGCAACCATCAATAAA
GGCTCCATAGTGTGGCAAGAAGTTTTCGACGATAAGGCCA
AATTGGCCCCCGGGACTATAGTGGAAGTATGGAAAGACAG
TGCATATCCCGAAGAGTTGTCCAGAGTCACAGCATCCGGA
TTTCCCGTTATTCTGAGTGCTCCATGGTATCTCAATAGGA
TATCTTACGGACAGGACTGGAGGAAATACTATAAGGTTGA
GCCATTGGACTTTGGGGGCACACAAAAACAGAAGCAGCTC
TTTATAGGCGGGGAGGCCTGTTTGTGGGGTGAATATGTTG
ATGCCACCAATCTTACTCCCAGATTGTGGCCCAGAGCATC
CGCAGTAGGCGAGCGACTCTGGAGCTCCAAAGACGTTAGG
GATATGGACGATGCTTATGATCGGCTGACTCGACACCGGT
GTAGAATGGTAGAACGGGGCATAGCAGCTCAACCCCTTTA
TGCTGGGTATTGCAACCACGAAAATATGTGA
HEXO
(SEQ ID NO: 3)
ATGGAGCTTTGTGGACTTGGCCTTCCACGACCACCCATGC
TCTTGGCTTTGTTGCTGGCCACCCTTTTGGCAGCCATGCT
TGCCCTTCTCACACAGGTAGCACTCGTCGTACAGGTCGCT
GAGGCAGCAAGGGCTCCCAGCGTATCAGCAAAGCCCGGCC
CAGCACTCTGGCCATTGCCCCTTCTGGTGAAGATGACTCC
AAACCTGCTGCATTTGGCCCCCGAGAACTTCTACATCTCT
CATAGCCCTAACAGCACCGCCGGCCCTTCATGTACTCTCC
TTGAAGAAGCATTCAGACGATATCATGGGTATATCTTTGG
ATTCTATAAGTGGCACCATGAGCCAGCAGAGTTCCAGGCC
AAAACACAGGTGCAGCAACTGCTCGTATCCATTACTCTGC
AGTCTGAATGCGACGCATTCCCAAATATTTCATCTGACGA
GTCCTACACTCTTCTTGTAAAAGAGCCCGTTGCTGTCCTG
AAAGCAAATAGGGTTTGGGGAGCCTTGAGAGGATTGGAGA
CCTTCTCACAACTCGTTTATCAAGATTCCTATGGAACTTT
TACCATCAACGAGTCAACCATAATTGATTCCCCCAGATTC
TCACACCGGGGCATTCTGATAGATACATCCCGCCACTATC
TCCCCGTAAAAATTATACTTAAAACCCTTGATGCAATGGC
TTTCAACAAATTTAACGTGTTGCATTGGCACATCGTCGAC
GACCAGAGTTTTCCCTATCAAAGTATCACATTCCCCGAAC
TCTCTAATAAGGGTAGCTACAGTTTGTCCCATGTATACAC
ACCCAACGATGTACGGATGGTTATAGAGTATGCCAGGCTG
CGGGGTATACGAGTCCTTCCAGAGTTCGACACACCTGGCC
ACACATTGAGTTGGGGCAAGGGACAAAAGGACCTTCTTAC
ACCATGCTACAGCGGCTCTGAGCCCAGTGGGACATTCGGT
CCCATTAACCCTACACTCAACACTACCTACAGTTTTCTTA
CCACTTTTTTTAAGGAAATTTCTGAAGTCTTCCCTGACCA
ATTCATACATCTTGGCGGAGACGAAGTAGAGTTCAAATGC
TGGGAATCAAACCCTAAGATCCAGGACTTTATGAGACAAA
AAGGATTTGGCACCGATTTCAAAAAGCTGGAGTCCTTTTA
CATTCAGAAAGTTTTGGATATTATCGCAACCATCAATAAA
GGCTCCATAGTGTGGCAAGAAGTTTTCGACGATAAGGCCA
AATTGGCCCCCGGGACTATAGTGGAAGTATGGAAAGACAG
TGCATATCCCGAAGAGTTGTCCAGAGTCACAGCATCCGGA
TTTCCCGTTATTCTGAGTGCTCCATGGTATCTCAATAGGA
TATCTTACGGACAGGACTGGAGGAAATACTATAAGGTTGA
GCCATTGGACTTTGGGGGCACACAAAAACAGAAGCAGCTC
TTTATAGGCGGGGAGGCCTGTTTGTGGGGTGAATATGTTG
ATGCCACCAATCTTACTCCCAGATTGTGGCCCAGAGCATC
CGCAGTAGGCGAGCGACTCTGGAGCTCCAAAGACGTTAGG
GATATGGACGATGCTTATGATCGGCTGACTCGACACCGGT
GTAGAATGGTAGAACGGGGCATAGCAGCTCAACCCCTTTA
TGCTGGGTATTGCAACCACGAAAATATGCTGGGAGGGGGA
GGATCTGGCGGAGGCGGAAGTGGCGGCGGAGGATCAGGGG
GGGGAGGCTCTCTGAGAAAGCTGCGGAAGCGGCTGCTGCT
GAGGAAGCTGAGAAAAAGACTGCTGTGA
IDUFA2
(SEQ ID NO: 4)
ATGCGGCCCCTGCGGCCTAGAGCCGCCCTGCTGGCTCTCC
TGGCTTCTCTGCTGGCCGCTCCCCCTGTCGCCCCTGCCGA
AGCCCCCCACCTGGTGCAGGTGGACGCCGCCAGAGCCCTG
TGGCCCCTGAGGCGGTTCTGGCGGAGCACCGGCTTTTGCC
CCCCTCTGCCCCACAGCCAGGCCGACCAGTACGTGCTGTC
CTGGGACCAGCAGCTGAACCTGGCCTACGTGGGCGCCGTG
CCCCACCGGGGCATCAAGCAGGTGCGGACCCACTGGCTGC
TGGAACTGGTGACCACCCGGGGCAGCACCGGCAGGGGCCT
GAGCTACAACTTCACCCACCTGGACGGCTACCTGGACCTG
CTGCGGGAGAACCAGCTGCTGCCCGGCTTCGAGCTGATGG
GCAGCGCCAGCGGCCACTTCACCGACTTCGAGGACAAGCA
GCAGGTGTTCGAGTGGAAGGACCTGGTGTCCAGCCTGGCT
CGCCGGTACATCGGCAGATACGGCCTGGCCCACGTGAGCA
AGTGGAACTTCGAGACCTGGAACGAGCCCGACCACCACGA
CTTCGACAACGTGAGCATGACCATGCAGGGCTTTCTGAAC
TACTACGACGCCTGCAGCGAGGGGCTGAGAGCCGCCAGCC
CTGCCCTGAGACTGGGCGGACCCGGCGACAGCTTCCACAC
CCCCCCCAGAAGCCCCCTGAGCTGGGGCCTGCTGCGGCAC
TGCCACGACGGCACCAATTTCTTCACCGGCGAGGCCGGCG
TGCGGCTGGACTACATCAGCCTGCACCGGAAGGGCGCCAG
AAGCAGCATCAGCATCCTGGAACAGGAAAAGGTCGTCGCT
CAGCAGATCCGGCAGCTGTTCCCCAAGTTCGCCGACACCC
CCATCTACAACGACGAGGCCGACCCCCTGGTGGGCTGGTC
TCTGCCCCAGCCTTGGAGGGCCGACGTGACCTACGCCGCC
ATGGTGGTGAAGGTGATCGCCCAGCACCAGAACCTGCTGC
TGGCCAACACCACCTCCGCCTTCCCTTACGCCCTGCTGTC
CAACGACAACGCCTTCCTGAGCTACCACCCCCACCCCTTC
GCCCAGCGGACCCTGACCGCCCGGTTCCAGGTGAACAACA
CCAGACCCCCCCACGTGCAGCTGCTGCGGAAGCCCGTGCT
GACCGCCATGGGGCTGCTGGCCCTGCTGGACGAGGAACAG
CTGTGGGCCGAGGTGTCCCAGGCCGGCACCGTGCTGGACA
GCAACCACACCGTGGGCGTGCTGGCTAGCGCCCACAGACC
TCAGGGCCCTGCCGATGCCTGGAGAGCCGCCGTGCTGATC
TACGCCAGCGACGACACCAGAGCCCACCCCAACCGGTCCG
TGGCCGTGACCCTGCGGCTGAGAGGCGTGCCTCCCGGCCC
TGGCCTGGTGTACGTGACCAGATACCTGGACAACGGCCTG
TGCAGCCCCGACGGCGAGTGGCGGAGGCTGGGCAGGCCCG
TGTTCCCCACCGCCGAGCAGTTCCGGCGGATGAGAGCCGC
CGAGGACCCCGTGGCCGCTGCCCCTAGACCTCTGCCTGCC
GGCGGACGGCTGACCCTGAGACCCGCCCTGAGGCTGCCCA
GCCTGCTGCTGGTGCACGTGTGCGCCAGGCCCGAGAAGCC
CCCAGGCCAGGTGACCCGGCTGCGCGCCCTGCCTCTGACC
CAGGGCCAGCTGGTGCTGGTGTGGAGCGACGAGCACGTGG
GCAGCAAGTGCCTGTGGACCTACGAGATCCAGTTCAGCCA
GGACGGCAAGGCCTACACCCCCGTGAGCCGGAAGCCCAGC
ACCTTCAACCTGTTCGTGTTCAGCCCCGACACAGGCGCCG
TGAGCGGCAGCTACAGAGTGCGGGCCCTGGACTACTGGGC
TCGCCCTGGCCCCTTCAGCGACCCCGTGCCCTACCTGGAA
GTGCCCGTGCCCAGAGGCCCTCCCAGCCCCGGCAACCCCC
TGGGAGGGGGAGGATCTGGCGGAGGCGGAAGTGGCGGCGG
AGGATCAGGGGGGGGAGGCTCTCTGAGAAAGCTGCGGAAG
CGGCTGCTGCTGAGGAAGCTGAGAAAAAGACTGCTGTGA
IDUAF1
(SEQ ID NO: 5)
ATGCGGCCCCTGCGGCCTAGAGCCGCCCTGCTGGCTCTCC
TGGCTTCTCTGCTGGCCGCTCCCCCTGTCGCCCCTGCCGA
AGCCCCCCACCTGGTGCAGGTGGACGCCGCCAGAGCCCTG
TGGCCCCTGAGGCGGTTCTGGCGGAGCACCGGCTTTTGCC
CCCCTCTGCCCCACAGCCAGGCCGACCAGTACGTGCTGTC
CTGGGACCAGCAGCTGAACCTGGCCTACGTGGGCGCCGTG
CCCCACCGGGGCATCAAGCAGGTGCGGACCCACTGGCTGC
TGGAACTGGTGACCACCCGGGGCAGCACCGGCAGGGGCCT
GAGCTACAACTTCACCCACCTGGACGGCTACCTGGACCTG
CTGCGGGAGAACCAGCTGCTGCCCGGCTTCGAGCTGATGG
GCAGCGCCAGCGGCCACTTCACCGACTTCGAGGACAAGCA
GCAGGTGTTCGAGTGGAAGGACCTGGTGTCCAGCCTGGCT
CGCCGGTACATCGGCAGATACGGCCTGGCCCACGTGAGCA
AGTGGAACTTCGAGACCTGGAACGAGCCCGACCACCACGA
CTTCGACAACGTGAGCATGACCATGCAGGGCTTTCTGAAC
TACTACGACGCCTGCAGCGAGGGGCTGAGAGCCGCCAGCC
CTGCCCTGAGACTGGGCGGACCCGGCGACAGCTTCCACAC
CCCCCCCAGAAGCCCCCTGAGCTGGGGCCTGCTGCGGCAC
TGCCACGACGGCACCAATTTCTTCACCGGCGAGGCCGGCG
TGCGGCTGGACTACATCAGCCTGCACCGGAAGGGCGCCAG
AAGCAGCATCAGCATCCTGGAACAGGAAAAGGTCGTCGCT
CAGCAGATCCGGCAGCTGTTCCCCAAGTTCGCCGACACCC
CCATCTACAACGACGAGGCCGACCCCCTGGTGGGCTGGTC
TCTGCCCCAGCCTTGGAGGGCCGACGTGACCTACGCCGCC
ATGGTGGTGAAGGTGATCGCCCAGCACCAGAACCTGCTGC
TGGCCAACACCACCTCCGCCTTCCCTTACGCCCTGCTGTC
CAACGACAACGCCTTCCTGAGCTACCACCCCCACCCCTTC
GCCCAGCGGACCCTGACCGCCCGGTTCCAGGTGAACAACA
CCAGACCCCCCCACGTGCAGCTGCTGCGGAAGCCCGTGCT
GACCGCCATGGGGCTGCTGGCCCTGCTGGACGAGGAACAG
CTGTGGGCCGAGGTGTCCCAGGCCGGCACCGTGCTGGACA
GCAACCACACCGTGGGCGTGCTGGCTAGCGCCCACAGACC
TCAGGGCCCTGCCGATGCCTGGAGAGCCGCCGTGCTGATC
TACGCCAGCGACGACACCAGAGCCCACCCCAACCGGTCCG
TGGCCGTGACCCTGCGGCTGAGAGGCGTGCCTCCCGGCCC
TGGCCTGGTGTACGTGACCAGATACCTGGACAACGGCCTG
TGCAGCCCCGACGGCGAGTGGCGGAGGCTGGGCAGGCCCG
TGTTCCCCACCGCCGAGCAGTTCCGGCGGATGAGAGCCGC
CGAGGACCCCGTGGCCGCTGCCCCTAGACCTCTGCCTGCC
GGCGGACGGCTGACCCTGAGACCCGCCCTGAGGCTGCCCA
GCCTGCTGCTGGTGCACGTGTGCGCCAGGCCCGAGAAGCC
CCCAGGCCAGGTGACCCGGCTGCGCGCCCTGCCTCTGACC
CAGGGCCAGCTGGTGCTGGTGTGGAGCGACGAGCACGTGG
GCAGCAAGTGCCTGTGGACCTACGAGATCCAGTTCAGCCA
GGACGGCAAGGCCTACACCCCCGTGAGCCGGAAGCCCAGC
ACCTTCAACCTGTTCGTGTTCAGCCCCGACACAGGCGCCG
TGAGCGGCAGCTACAGAGTGCGGGCCCTGGACTACTGGGC
TCGCCCTGGCCCCTTCAGCGACCCCGTGCCCTACCTGGAA
GTGCCCGTGCCCAGAGGCCCTCCCAGCCCCGGCAACCCCC
GATCGAGCGCTACGCGTGTCGACGAACAAAAACTCATCTC
AGAAGAGGATCTGATTGACATTTTGGAGCTGCGCAAGCTG
CGTAAGCGGCTCCTCCTGCGCAAGCTGCGTAAGCGGCTCC
TCTAG

### Example II

PS Gene Editing with a HEXO Construct to Treat Both Tay-Sachs and Sandhoff Diseases

Neurological symptoms may be treated as a result of the integrity of blood-brain barrier being impaired due to disease, exosomes, pinocytosis, extracellular pathway, and/or uncharacterized receptors.

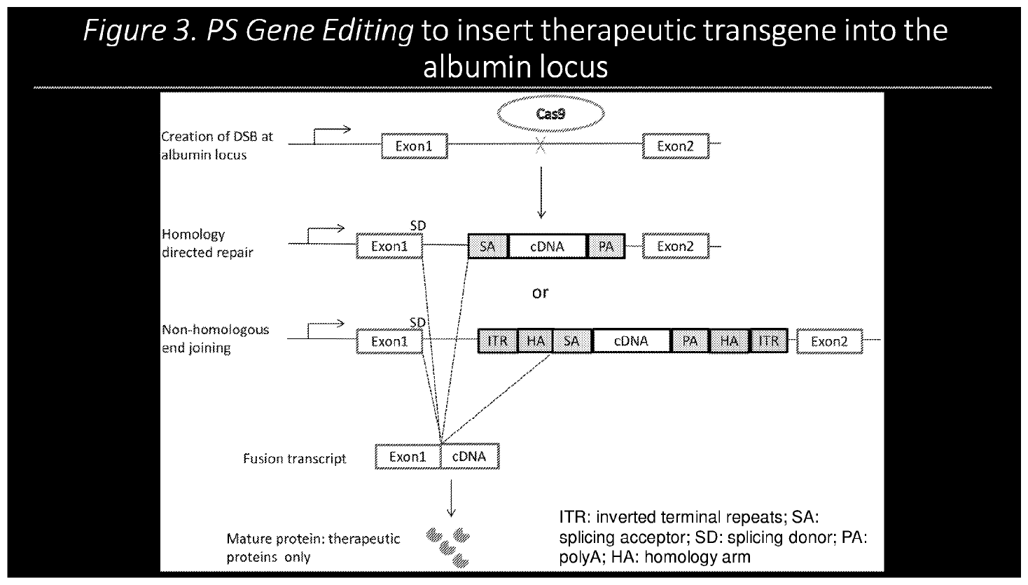

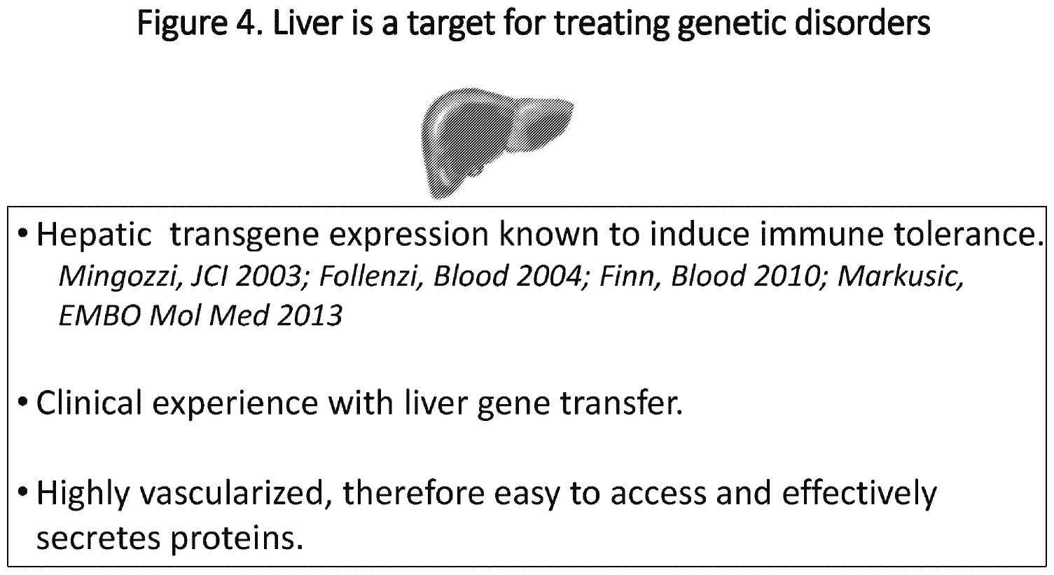

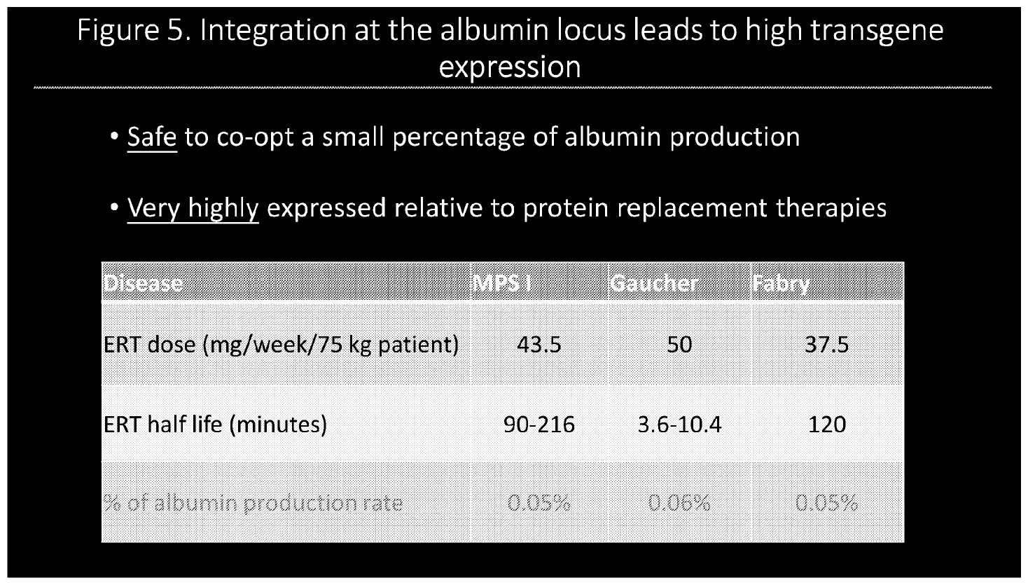

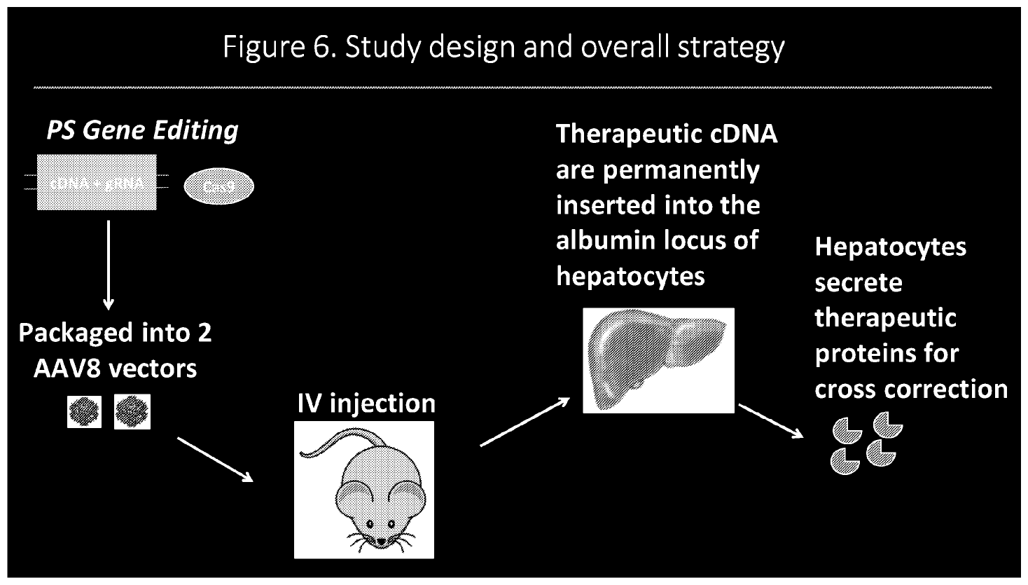

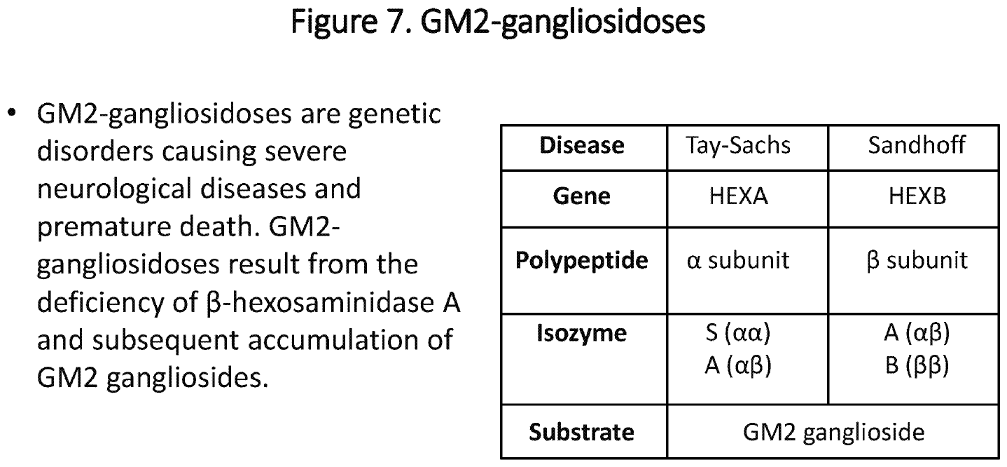

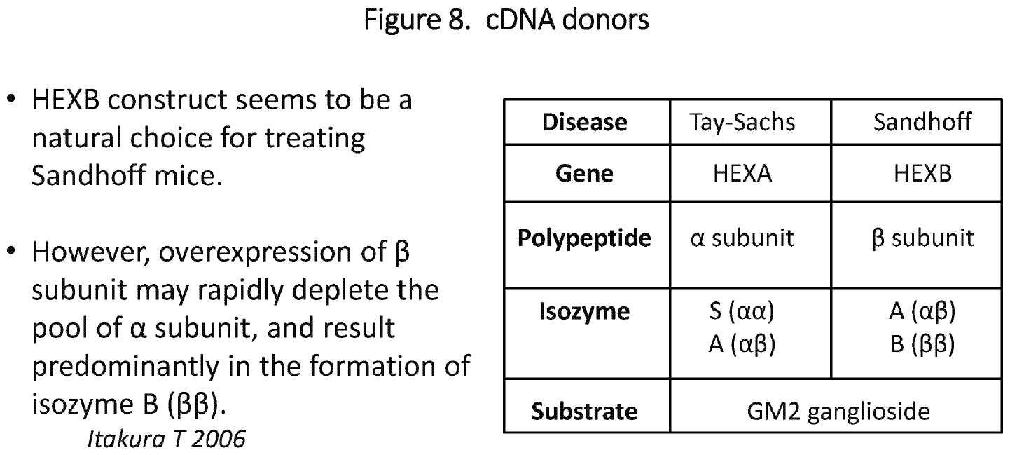

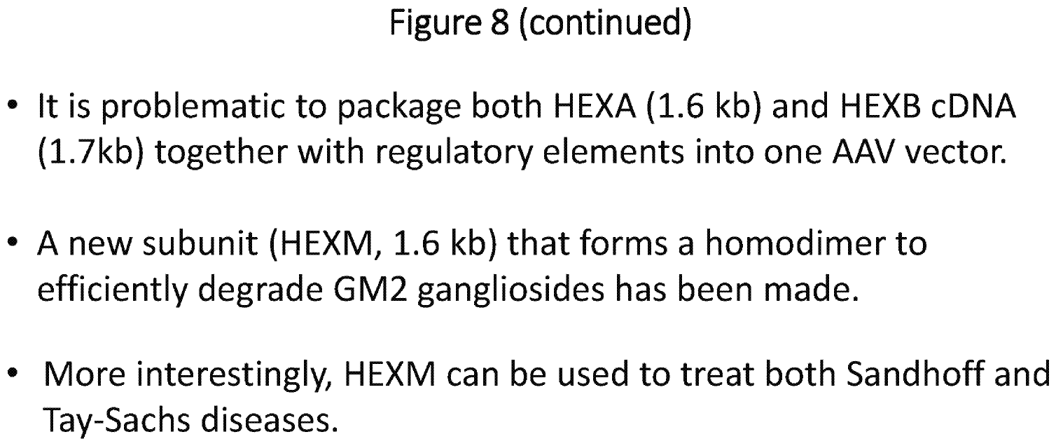

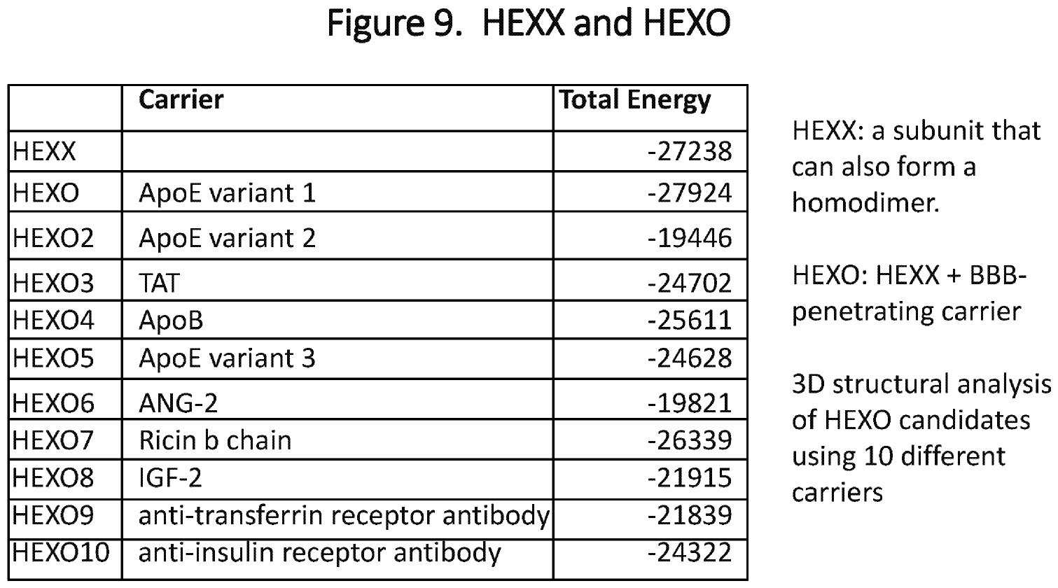

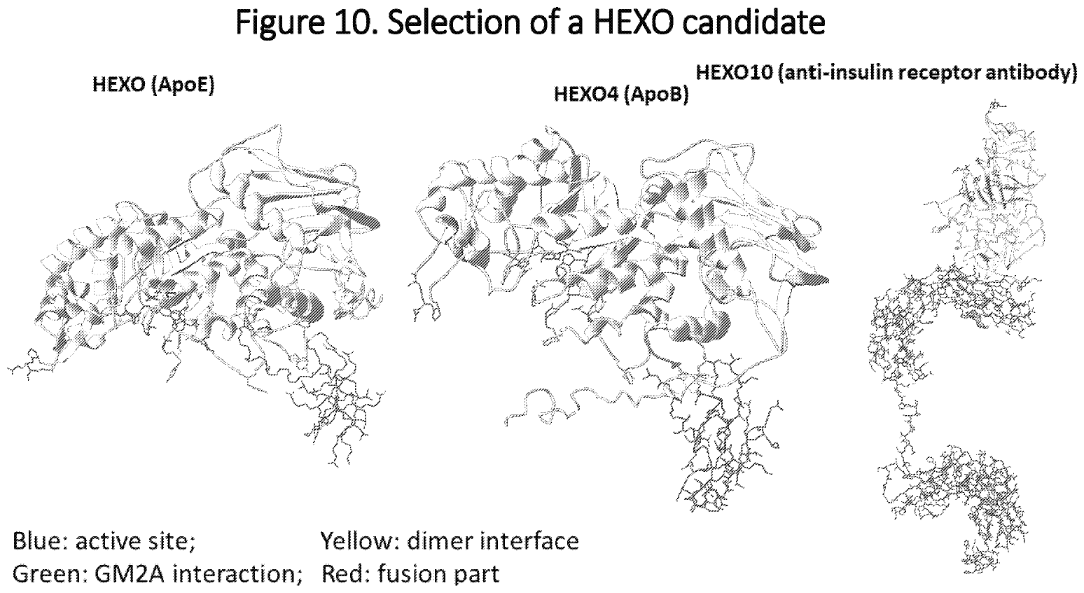

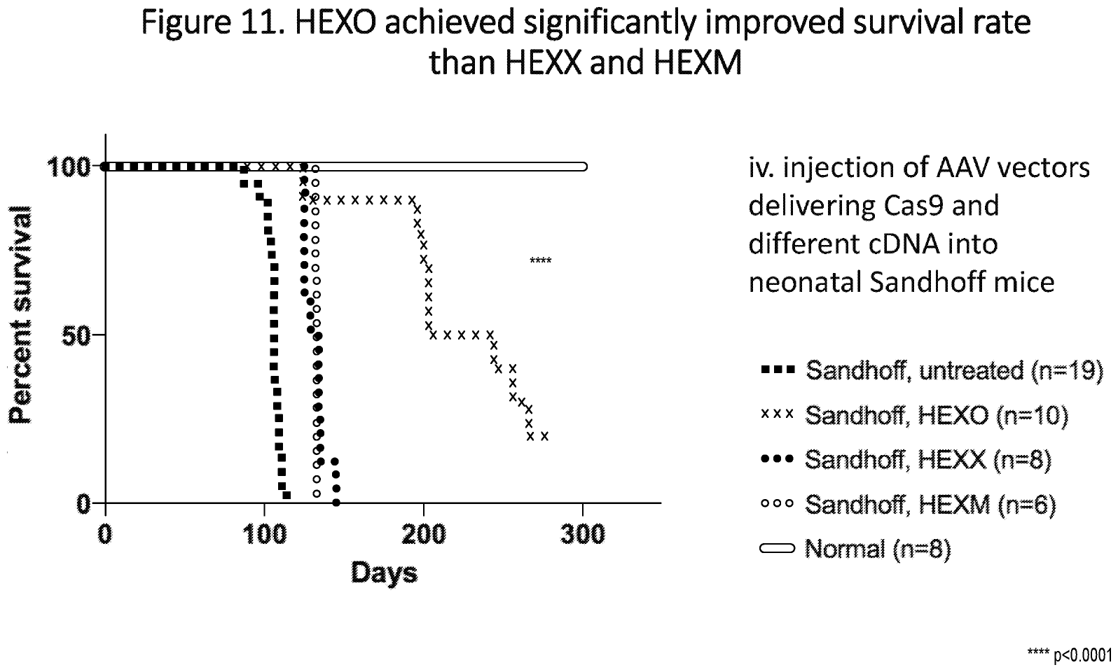

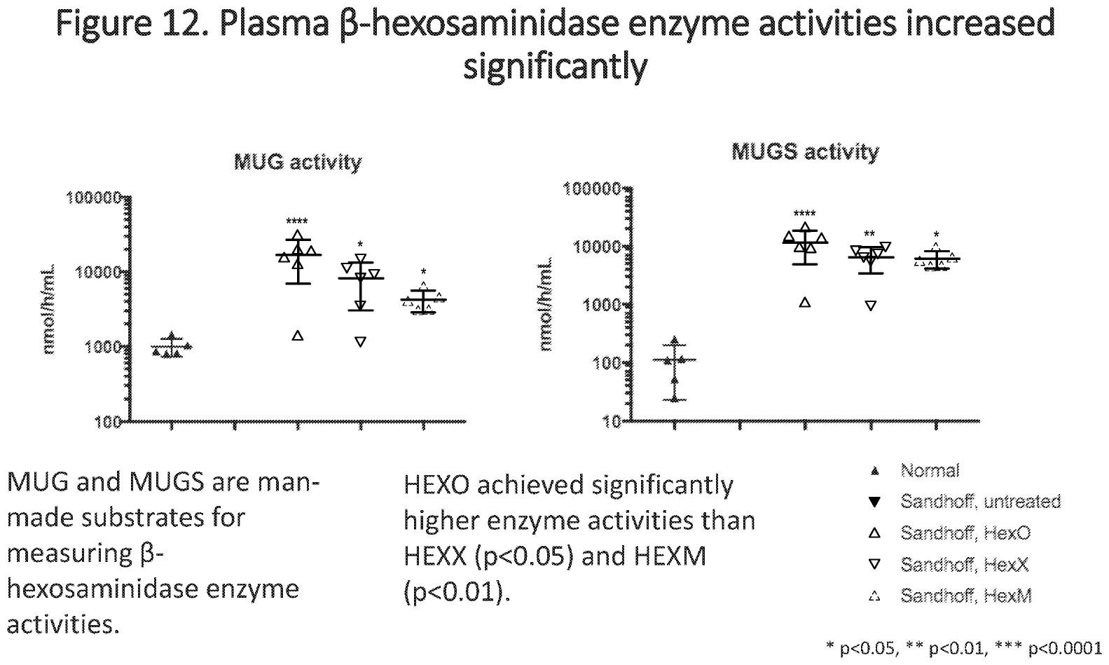

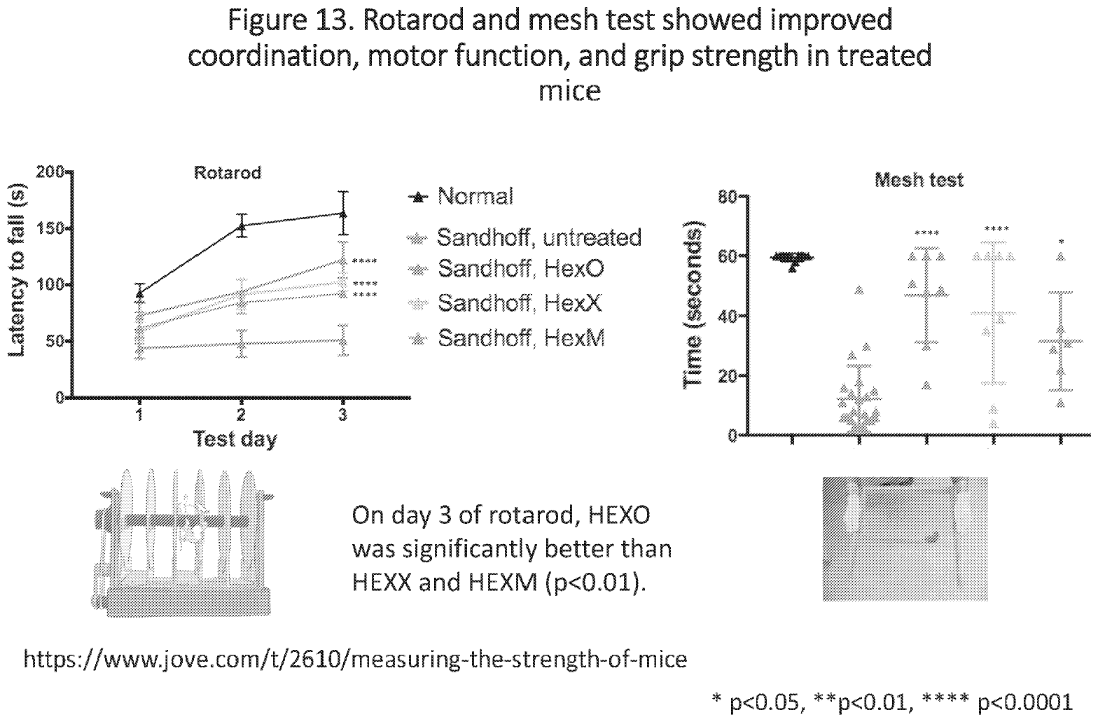

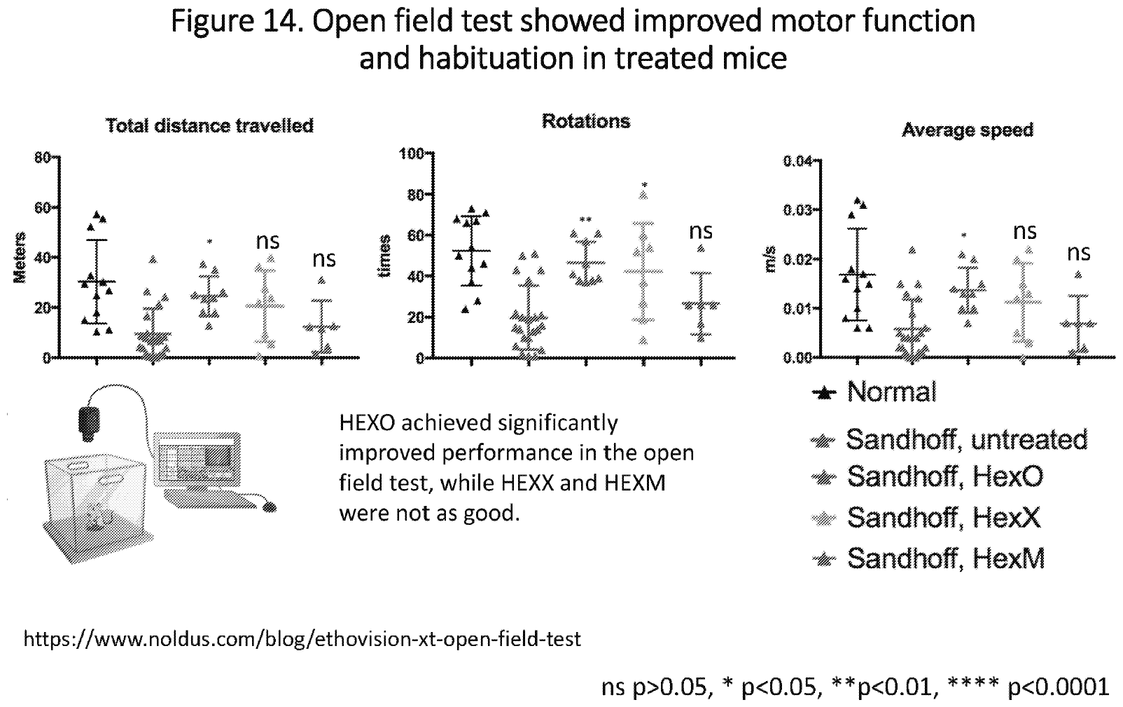

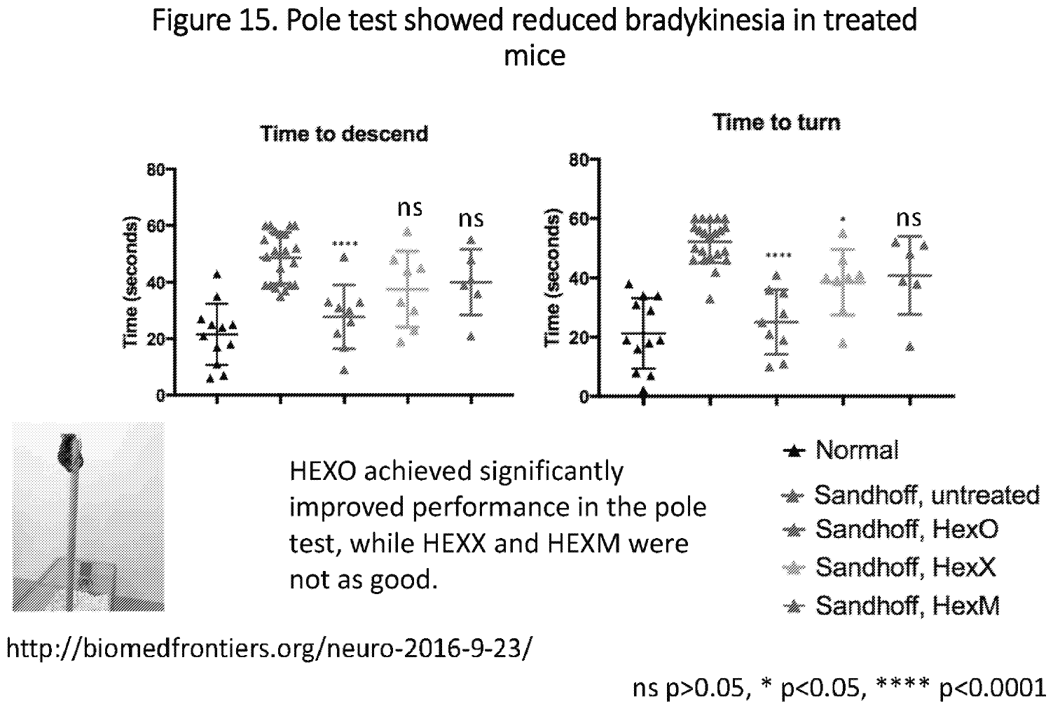

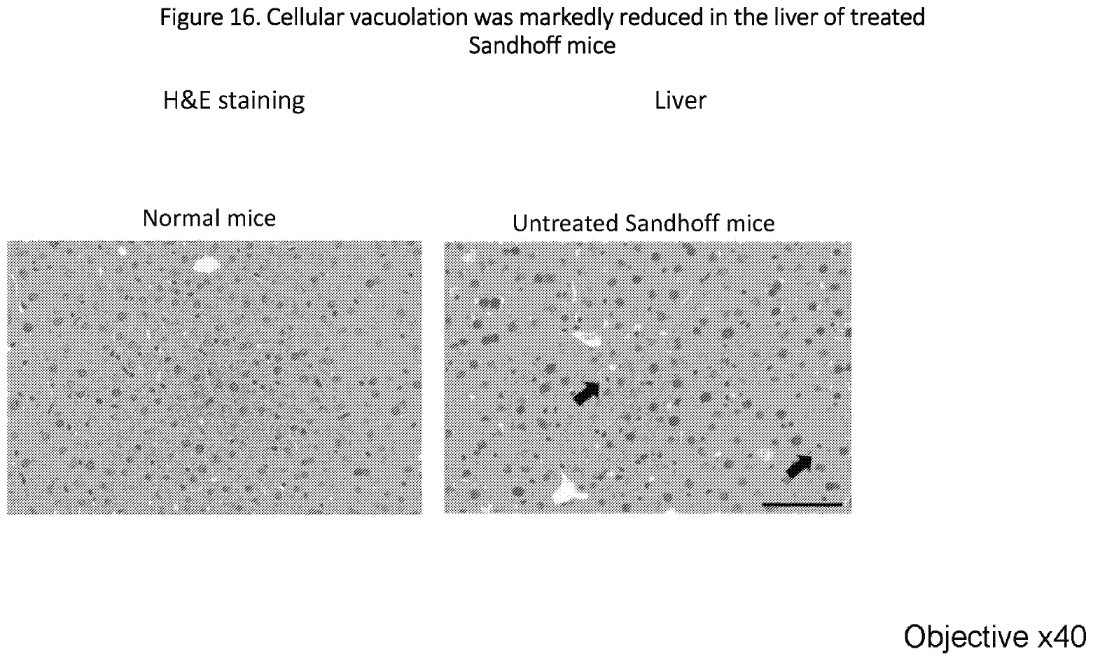

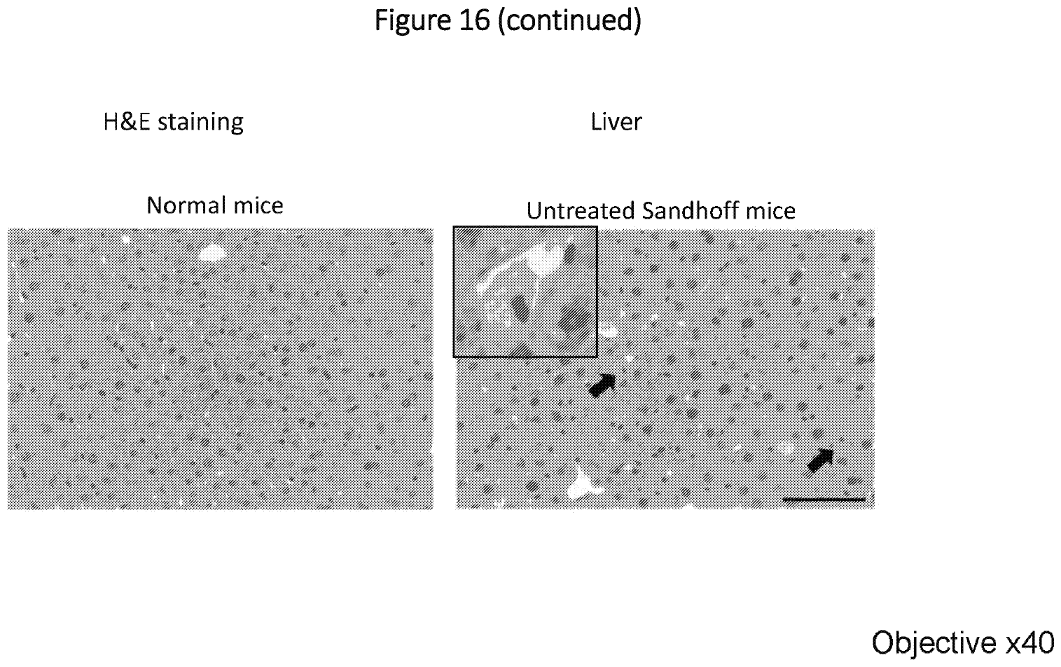

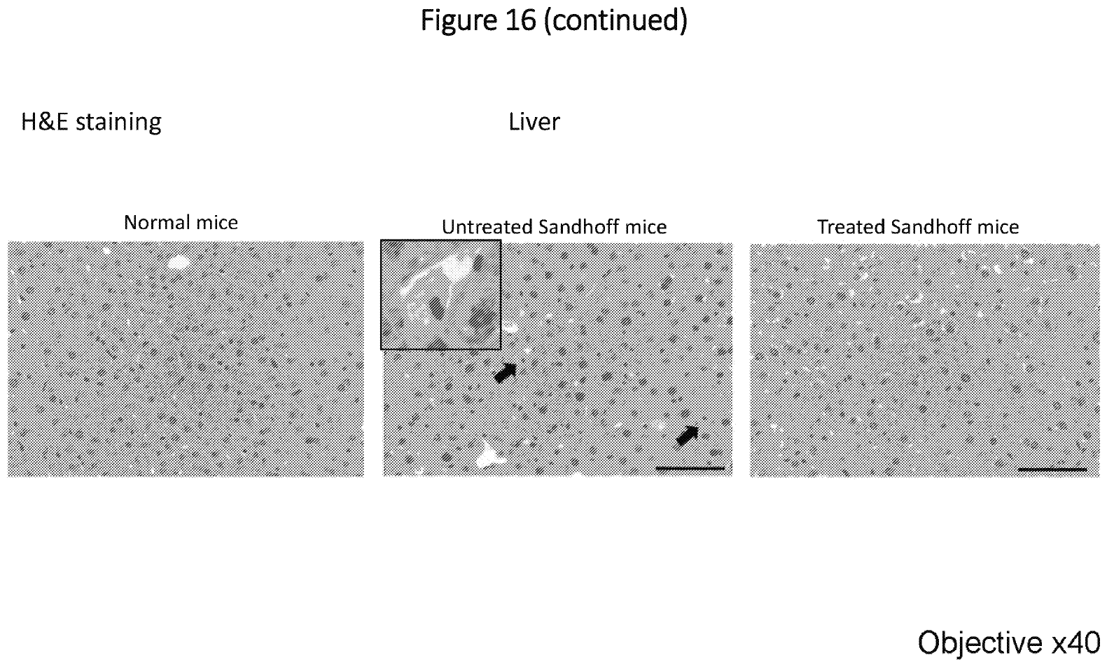

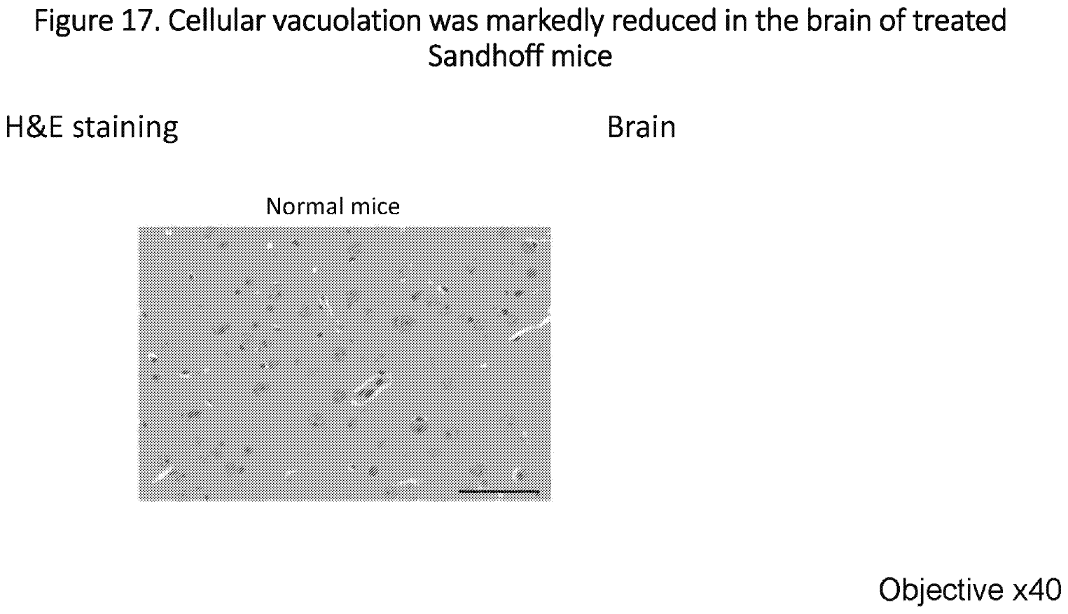

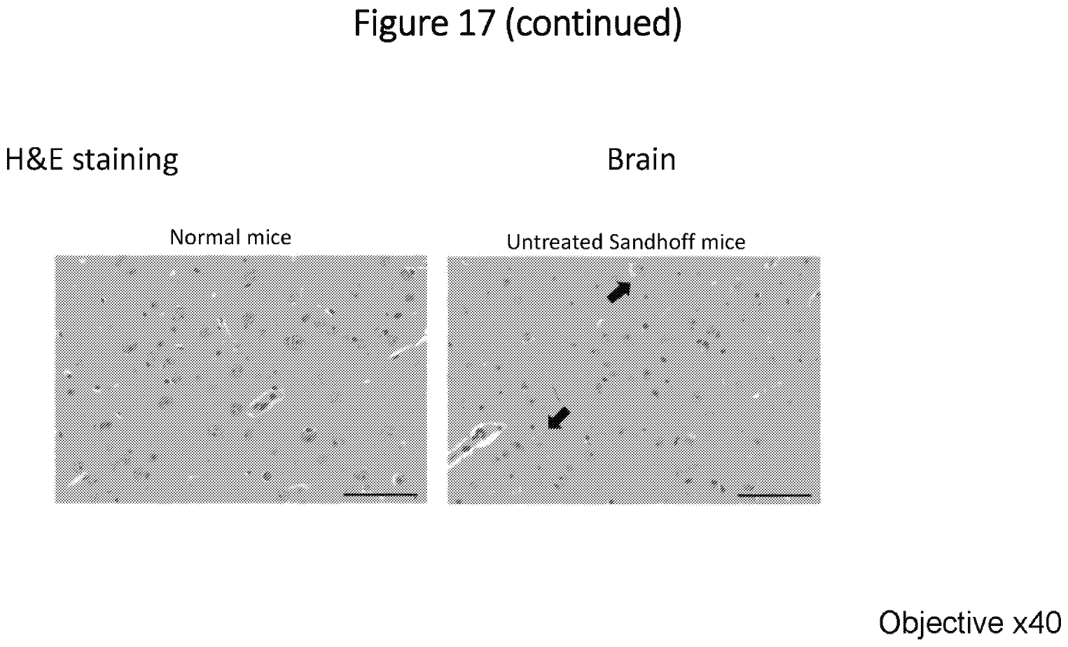

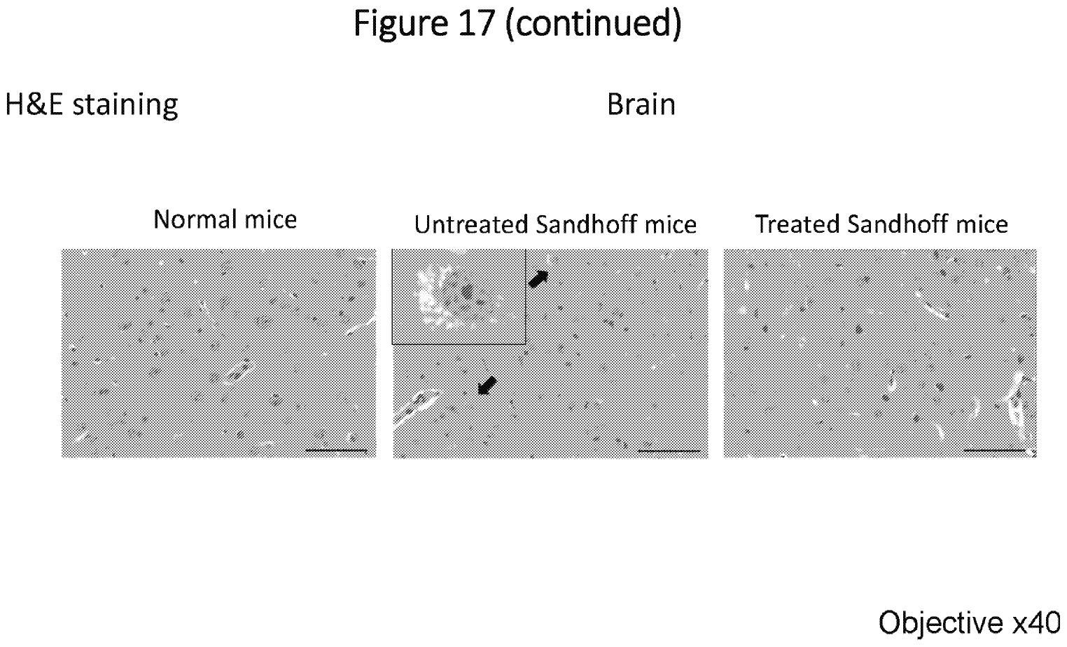

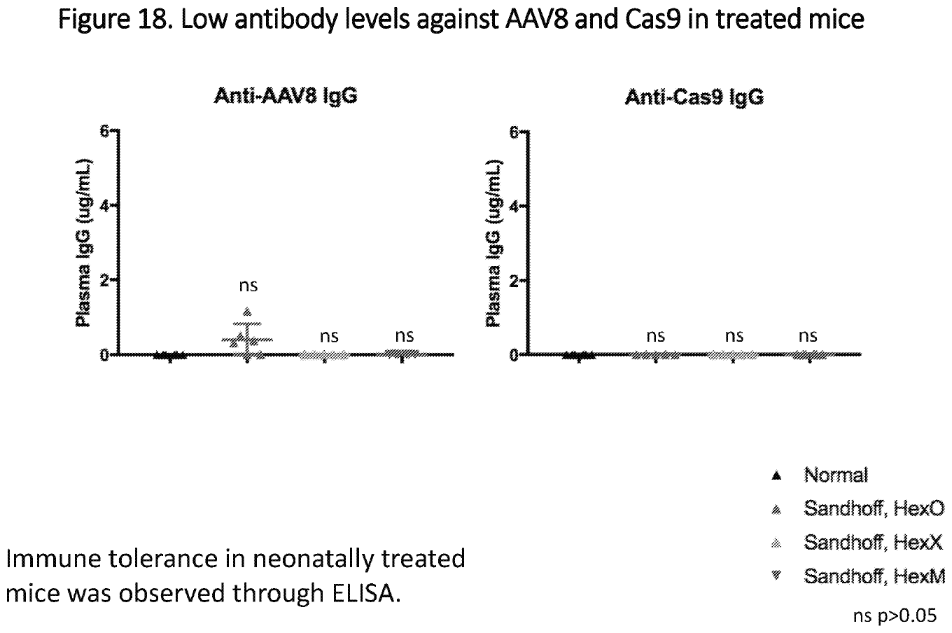

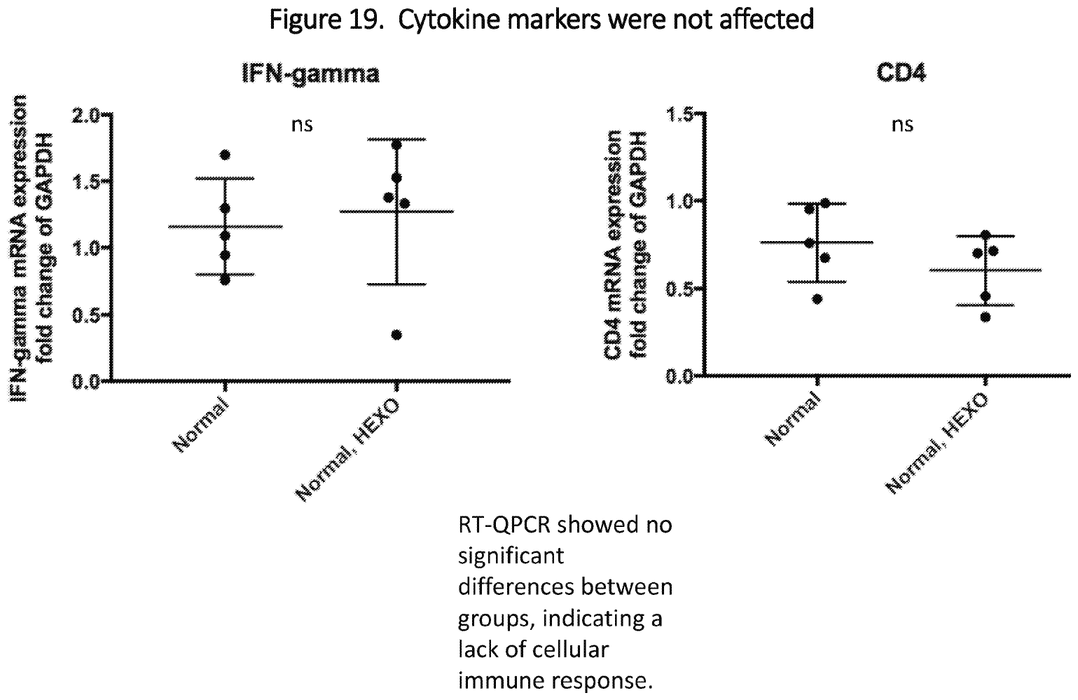

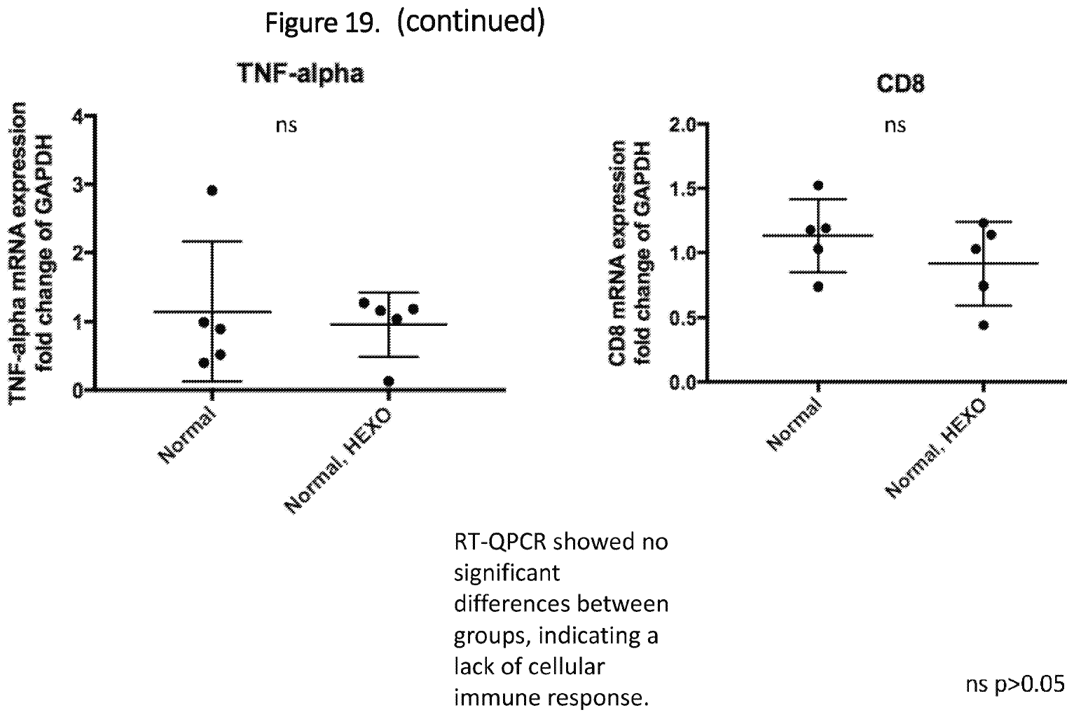

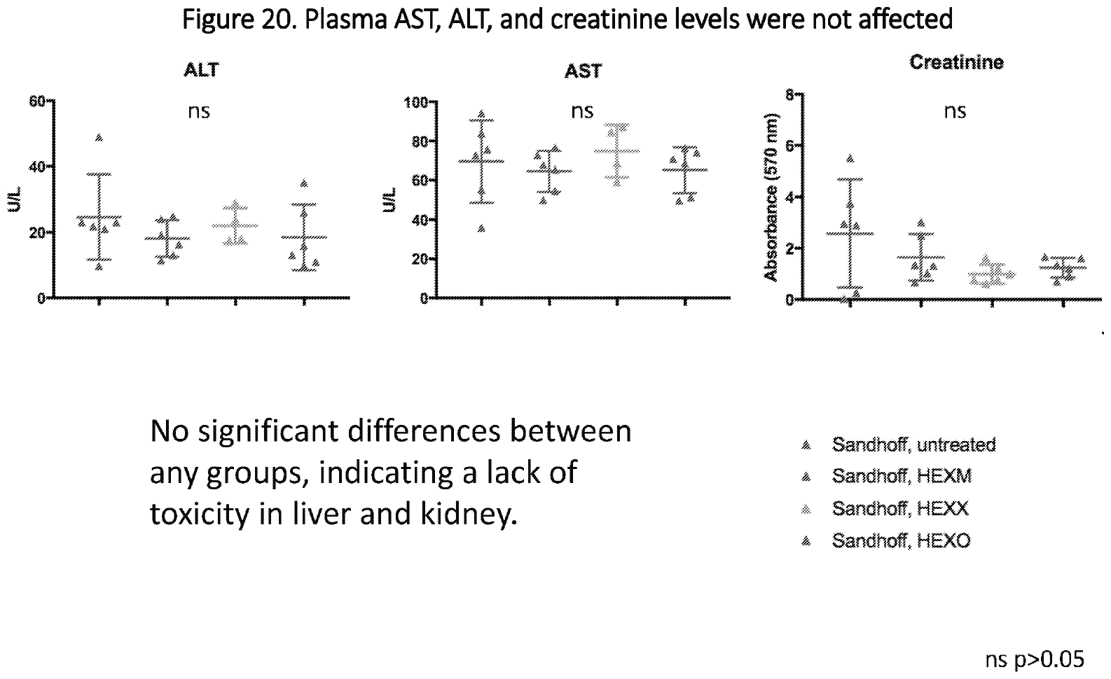

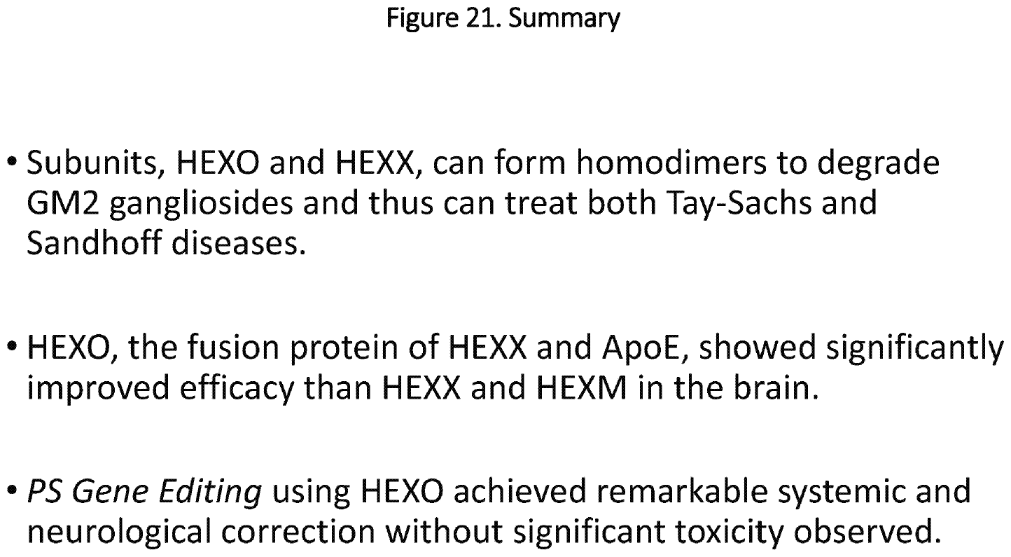

For studies related to PS Gene Editing with a HEXO construct to treat both Tay-Sachs and Sandhoff diseases, see FIGS. 3-21.

### References

Blanz, J., Stroobants, S., Lüllmann-Rauch, R., Morelle, W., Lüdemann, M., D'Hooge, R., Reuterwall, H., Michalski, J. C., Fogh, J., Andersson, C., et al. (2008). Reversal of peripheral and central neural storage and ataxia after recombinant enzyme replacement therapy in alpha-mannosidosis mice. Hum Mol Genet. 17(22), 3437-3445.
Böckenhoff A, Cramer S, Wölte P, Knieling S, Wohlenberg C, Gieselmann V, Galla H J, Matzner U. Comparison of five peptide vectors for improved brain delivery of the lysosomal enzyme arylsulfatase A. J Neurosci. 2014 Feb. 26; 34(9):3122-9.
Dunder, U., Kaartinen, V., Valtonen, P., Väänänen, E., Kosma, V. M., Heisterkamp, N., Groffen, J., Mononen, I. (2000). Enzyme replacement therapy in a mouse model of aspartylglycosaminuria. FASEB J. 14(2), 361-367.
El-Amouri S S, Dai M, Han J F, Brady R O, Pan D. Normalization and improvement of CNS deficits in mice with Hurler syndrome after long-term peripheral delivery of BBB-targeted iduronidase. Mol Ther. 2014 December; 22(12):2028-37.
Gleitz H F, Liao A Y, Cook J R, Rowlston S F, Forte G M, D'Souza Z, O'Leary C, Holley R J, Bigger B W. Brain-targeted stem cell gene therapy corrects mucopolysaccharidosis type II via multiple mechanisms. EMBO Mol Med. 2018 July; 10(7).
Kitakaze K, Mizutani Y, Sugiyama E, Tasaki C, Tsuji D, Maita N, Hirokawa T, Asanuma D, Kamiya M, Sato K, Setou M, Urano Y, Togawa T, Otaka A, Sakuraba H, Itoh K. Protease-resistant modified human β-hexosaminidase B ameliorates symptoms in GM2 gangliosidosis model. J Clin Invest. 2016 May 2; 126(5):1691-703.
Lee, W C., Courtenay, A., Troendle, F. J., Stallings-Mann, M. L., Dickey, C. A., DeLucia, M. W., Dickson, D. W., Eckman, C. B. (2005). Enzyme replacement therapy results in substantial improvements in early clinical phenotype in a mouse model of globoid cell leukodystrophy. FASEB J. 19(11), 1549-1551.
Matzner, U., Herbst, E., Hedayati, K. K., Lüllmann-Rauch, R., Wessig, C., Schröder, S., Eistrup, C., Moller, C., Fogh, J., Gieselmann, V. (2005). Enzyme replacement improves nervous system pathology and function in a mouse model for metachromatic leukodystrophy, Hum Mol Genet. 14(9), 1139-1152.
Matzner, U., Löllmann-Rauch, R., Stroobants, S., Andersson, C., Weigelt, C., Eistrup, C., Fogh, J., D'Hooge, R., Gieselmann, V. (2009). Enzyme replacement improves ataxic gait and central nervous system histopathology in a mouse model of metachromatic leukodystrophy. Mol Ther. 17(4), 600-606.
Pardridge W M. Blood-brain barrier delivery. Drug Discov Today. 2007 January; 12(1-2):54-61.
Polito, V. A., Abbondante, S., Polishchuk, R. S., Nusco, E., Salvia, R., Cosma, M. P. (2010). Correction of CNS defects in the MPSII mouse model via systemic enzyme replacement therapy. Hum Mol Genet. 19(24), 4871-4885.
Roces, D. P., Lüllmann-Rauch, R., Peng, J., Balducci, C., Andersson, C., Tollersrud, O., Fogh, J., Orlacchio, A., Beccari, T., Saftig, P., et al. (2004). Efficacy of enzyme replacement therapy in alpha-mannosidosis mice: a preclinical animal study. Hum Mol Genet. 13(18), 1979-1988.
Rozaklis, T., Beard, H., Hassiotis, S., Garcia, A. R., Tonini, M., Luck, A., Pan, J., Lamsa, J. C., Hopwood, J. J., Hemsley, K. M. (2011). Impact of high-dose, chemically modified sulfamidase on pathology in a murine model of MPS IIIA. Exp Neurol. 230(1), 123-130.
Tropak M B, Yonekawa S, Karumuthil-Melethil S, Thompson P, Wakarchuk W, Gray S J, Walia J S, Mark B L, Mahuran D. Construction of a hybrid β-hexosaminidase subunit capable of forming stable homodimers that hydrolyze GM2 ganglioside in vivo. Mol Ther Methods Clin Dev. 2016 Mar. 2; 3:15057.
Ueno M, Nakagawa T, Wu B, Onodera M, Huang C L, Kusaka T, Araki N, Sakamoto H. Transporters in the brain endothelial barrier. Curr Med Chem. 2010; 17(12):1125-38.
Vogler, C., Levy, B., Grubb, J. H., Galvin, N., Tan, Y., Kakkis, E., Pavloff, N., Sly, W. S. (2005). Overcoming the blood—brain barrier with high-dose enzyme replacement therapy in murine mucopolysaccharidosis VII. Proc Natl Acad Sci USA. 102(41), 14777-14782.
Wang D, El-Amouri S S, Dai M, Kuan C Y, Hui D Y, Brady R O, Pan D. Engineering a lysosomal enzyme with a derivative of receptor-binding domain of apoE enables delivery across the blood-brain barrier. Proc Natl Acad Sci USA. 2013 Feb. 19; 110(8):2999-3004.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A nucleic acid vector encoding a fusion polypeptide comprising a modified hexosaminidase beta-subunit that forms homodimers, a linker and an ApoE peptide that allows for transport across the blood brain barrier.

2. The vector of claim 1 which is a plasmid or a viral vector.

3. (canceled)

4. The vector of claim 1 3 which encodes a rnultimer of the ApoE peptide.

5. The vector of claim 1 wherein the hexoseaminidase beta-subunit is encoded by SEQ ID NO:1, SEQ ID NO:2 or SEQ ID NO:3 or has at least 90% amino acid sequence identity to the hexoseaminidase beta-subunit is encoded by SEQ ID NO:1, SEQ ID NO:2 or SEQ ID NO:3 and includes one or more of G, S, E or P at residue 312, 313, 314 or 315, respectively, S, G or T at residue 316, 317 or 318, respectively, or N or R at residue 452 or 453, respectively, or any combination thereof.

6. The vector of claim 1 wherein the vector comprises a nucleotide sequence having at least 80%, 90% or 95% nucleic acid sequence identity to SEQ ID NO:2 or SEQ ID NO:3.

7. The vector of claim 1 wherein the linker comprises X₁DX₂X₃E, wherein X₁, X₂ or X₃ individually is I, L, V, A or G, wherein the linker comprises X₁X₄X₂X₃X₅, wherein X₄ or X₅ individually is D, E, R, K, N or Q, wherein the linker comprises IDLE, wherein the linker comprises X₆X₇X₈X₉X₁₀, wherein each of X₆, X₇, X₈ and X₉ individually is I, L, V, A or G and wherein X₁₀ is S or T or X₆X₇X₁₀, X₆X₁₀X₁₀, wherein the linker comprises GGGGS, GGS, GSS, or GSSSSSS or wherein the linker comprises LGGGGSGGGGSGGGGSGGGGS.

8-12. (canceled)

13. The vector of claim 1 wherein the ApoE peptide comprises X₁₁X₁₂X₁₃X₁₄X₁₅ X₁₆X₁₇X₁₈ X₁₉, wherein each of X₁₁, X₁₄, X₁₅, X₁₈, and X₁₉ individually is I, L, V, A or G, and wherein each of X₁₂, X₁₃, X₁₅ X₁₆, and X₁₇ is R, K or H or wherein the ApoE peptide comprises LRKLRKRLL.

14. (canceled)

15. The vector of claim 1 which is an adeno-associated virus vector, an adenovirus vector, a lentivirus vector, a herpesvirus vector or a retroviral vector.

16. The vector of claim 1 which comprises an open reading frame for the fusion polypeptide which operably links an open reading frame for the modified hexosaminidase beta-subunit that forms homodimers via the nucleotide sequence encoding the linker to the open reading frame for the ApoE peptide,

17-18. (canceled)

19. A cell comprising the vector of claim 1.

20-21. (canceled)

22. A polypeptide encoded by the vector of claim 1.

23-24. (canceled)

25. A method to prevent, inhibit or treat one or more symptoms of GM2-gangliosidosis in a mammal, comprising: administering to the mammal an effective amount of a composition comprising the vector of claim 1.

27. The method of claim 26 wherein the human has Sandhoff disease or Tay-Sachs disease.

28. The method of claim 25 wherein the composition is systemically administered or injected.

29. (canceled)

30. The method of claim 25 wherein the composition is administered to the central nervous system or the cerebrospinal fluid.

31. (canceled)

32. The method of claim 25 wherein the composition comprises particles or liposomes comprising the vector.

33. The method of claim 32 wherein the vector comprises RNA.

34. The method of claim 32 wherein the particles are nanoparticles.

35. A method to prevent, inhibit or treat one or more symptoms of GM2-gangliosidosis in a mammal, comprising: administering to the mammal an effective amount of a composition comprising a polypeptide comprising a modified hexosaminidase beta-subunit that forms homodimers, a linker and an ApoE peptide that allows for transport across the blood brain barrier.


SEQUENCE LISTING (51 sequences)

SEQ ID NO: 1 - DNA, 1671 bp, Artificial Sequence
  A synthetic sequence
atggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtgggtctgagccctctggcacctttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttaaaccgtattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 2 - DNA, 1671 bp, Artificial Sequence
  A synthetic sequence
atggagctttgtggacttggccttccacgaccacccatgctcttggctttgttgctggccacccttttggcagccatgcttgcccttctcacacaggtagcactcgtcgtacaggtcgctgaggcagcaagggctcccagcgtatcagcaaagcccggcccagcactctggccattgccccttctggtgaagatgactccaaacctgctgcatttggcccccgagaacttctacatctctcatagccctaacagcaccgccggcccttcatgtactctccttgaagaagcattcagacgatatcatgggtatatctttggattctataagtggcaccatgagccagcagagttccaggccaaaacacaggtgcagcaactgctcgtatccattactctgcagtctgaatgcgacgcattcccaaatatttcatctgacgagtcctacactcttcttgtaaaagagcccgttgctgtcctgaaagcaaatagggtttggggagccttgagaggattggagaccttctcacaactcgtttatcaagattcctatggaacttttaccatcaacgagtcaaccataattgattcccccagattctcacaccggggcattctgatagatacatcccgccactatctccccgtaaaaattatacttaaaacccttgatgcaatggctttcaacaaatttaacgtgttgcattggcacatcgtcgacgaccagagttttccctatcaaagtatcacattccccgaactctctaataagggtagctacagtttgtcccatgtatacacacccaacgatgtacggatggttatagagtatgccaggctgcggggtatacgagtccttccagagttcgacacacctggccacacattgagttggggcaagggacaaaaggaccttcttacaccatgctacagcggctctgagcccagtgggacattcggtcccattaaccctacactcaacactacctacagttttcttaccactttttttaaggaaatttctgaagtcttccctgaccaattcatacatcttggcggagacgaagtagagttcaaatgctgggaatcaaaccctaagatccaggactttatgagacaaaaaggatttggcaccgatttcaaaaagctggagtccttttacattcagaaagttttggatattatcgcaaccatcaataaaggctccatagtgtggcaagaagttttcgacgataaggccaaattggcccccgggactatagtggaagtatggaaagacagtgcatatcccgaagagttgtccagagtcacagcatccggatttcccgttattctgagtgctccatggtatctcaataggatatcttacggacaggactggaggaaatactataaggttgagccattggactttgggggcacacaaaaacagaagcagctctttataggcggggaggcctgtttgtggggtgaatatgttgatgccaccaatcttactcccagattgtggcccagagcatccgcagtaggcgagcgactctggagctccaaagacgttagggatatggacgatgcttatgatcggctgactcgacaccggtgtagaatggtagaacggggcatagcagctcaacccctttatgctgggtattgcaaccacgaaaatatgtga

SEQ ID NO: 3 - DNA, 1788 bp, Artificial Sequence
  A synthetic sequence
atggagctttgtggacttggccttccacgaccacccatgctcttggctttgttgctggccacccttttggcagccatgcttgcccttctcacacaggtagcactcgtcgtacaggtcgctgaggcagcaagggctcccagcgtatcagcaaagcccggcccagcactctggccattgccccttctggtgaagatgactccaaacctgctgcatttggcccccgagaacttctacatctctcatagccctaacagcaccgccggcccttcatgtactctccttgaagaagcattcagacgatatcatgggtatatctttggattctataagtggcaccatgagccagcagagttccaggccaaaacacaggtgcagcaactgctcgtatccattactctgcagtctgaatgcgacgcattcccaaatatttcatctgacgagtcctacactcttcttgtaaaagagcccgttgctgtcctgaaagcaaatagggtttggggagccttgagaggattggagaccttctcacaactcgtttatcaagattcctatggaacttttaccatcaacgagtcaaccataattgattcccccagattctcacaccggggcattctgatagatacatcccgccactatctccccgtaaaaattatacttaaaacccttgatgcaatggctttcaacaaatttaacgtgttgcattggcacatcgtcgacgaccagagttttccctatcaaagtatcacattccccgaactctctaataagggtagctacagtttgtcccatgtatacacacccaacgatgtacggatggttatagagtatgccaggctgcggggtatacgagtccttccagagttcgacacacctggccacacattgagttggggcaagggacaaaaggaccttcttacaccatgctacagcggctctgagcccagtgggacattcggtcccattaaccctacactcaacactacctacagttttcttaccactttttttaaggaaatttctgaagtcttccctgaccaattcatacatcttggcggagacgaagtagagttcaaatgctgggaatcaaaccctaagatccaggactttatgagacaaaaaggatttggcaccgatttcaaaaagctggagtccttttacattcagaaagttttggatattatcgcaaccatcaataaaggctccatagtgtggcaagaagttttcgacgataaggccaaattggcccccgggactatagtggaagtatggaaagacagtgcatatcccgaagagttgtccagagtcacagcatccggatttcccgttattctgagtgctccatggtatctcaataggatatcttacggacaggactggaggaaatactataaggttgagccattggactttgggggcacacaaaaacagaagcagctctttataggcggggaggcctgtttgtggggtgaatatgttgatgccaccaatcttactcccagattgtggcccagagcatccgcagtaggcgagcgactctggagctccaaagacgttagggatatggacgatgcttatgatcggctgactcgacaccggtgtagaatggtagaacggggcatagcagctcaacccctttatgctgggtattgcaaccacgaaaatatgctgggagggggaggatctggcggaggcggaagtggcggcggaggatcagggggcggaggctctctgagaaagctgcggaagcggctgctgctgaggaagctgagaaaaagactgctgtga

SEQ ID NO: 4 - DNA, 2079 bp, Artificial Sequence
  A synthetic sequence
atgcggcccctgcggcctagagccgccctgctggctctcctggcttctctgctggccgctccccctgtcgcccctgccgaagccccccacctggtgcaggtggacgccgccagagccctgtggcccctgaggcggttctggcggagcaccggcttttgcccccctctgccccacagccaggccgaccagtacgtgctgtcctgggaccagcagctgaacctggcctacgtgggcgccgtgccccaccggggcatcaagcaggtgcggacccactggctgctggaactggtgaccacccggggcagcaccggcaggggcctgagctacaacttcacccacctggacggctacctggacctgctgcgggagaaccagctgctgcccggcttcgagctgatgggcagcgccagcggccacttcaccgacttcgaggacaagcagcaggtgttcgagtggaaggacctggtgtccagcctggctcgccggtacatcggcagatacggcctggcccacgtgagcaagtggaacttcgagacctggaacgagcccgaccaccacgacttcgacaacgtgagcatgaccatgcagggctttctgaactactacgacgcctgcagcgaggggctgagagccgccagccctgccctgagactgggcggacccggcgacagcttccacaccccccccagaagccccctgagctggggcctgctgcggcactgccacgacggcaccaatttcttcaccggcgaggccggcgtgcggctggactacatcagcctgcaccggaagggcgccagaagcagcatcagcatcctggaacaggaaaaggtcgtcgctcagcagatccggcagctgttccccaagttcgccgacacccccatctacaacgacgaggccgaccccctggtgggctggtctctgccccagccttggagggccgacgtgacctacgccgccatggtggtgaaggtgatcgcccagcaccagaacctgctgctggccaacaccacctccgccttcccttacgccctgctgtccaacgacaacgccttcctgagctaccacccccaccccttcgcccagcggaccctgaccgcccggttccaggtgaacaacaccagacccccccacgtgcagctgctgcggaagcccgtgctgaccgccatggggctgctggccctgctggacgaggaacagctgtgggccgaggtgtcccaggccggcaccgtgctggacagcaaccacaccgtgggcgtgctggctagcgcccacagacctcagggccctgccgatgcctggagagccgccgtgctgatctacgccagcgacgacaccagagcccaccccaaccggtccgtggccgtgaccctgcggctgagaggcgtgcctcccggccctggcctggtgtacgtgaccagatacctggacaacggcctgtgcagccccgacggcgagtggcggaggctgggcaggcccgtgttccccaccgccgagcagttccggcggatgagagccgccgaggaccccgtggccgctgcccctagacctctgcctgccggcggacggctgaccctgagacccgccctgaggctgcccagcctgctgctggtgcacgtgtgcgccaggcccgagaagcccccaggccaggtgacccggctgcgcgccctgcctctgacccagggccagctggtgctggtgtggagcgacgagcacgtgggcagcaagtgcctgtggacctacgagatccagttcagccaggacggcaaggcctacacccccgtgagccggaagcccagcaccttcaacctgttcgtgttcagccccgacacaggcgccgtgagcggcagctacagagtgcgggccctggactactgggctcgccctggccccttcagcgaccccgtgccctacctggaagtgcccgtgcccagaggccctcccagccccggcaaccccctgggagggggaggatctggcggaggcggaagtggcggcggaggatcagggggcggaggctctctgagaaagctgcggaagcggctgctgctgaggaagctgagaaaaagactgctgtga

SEQ ID NO: 5 - DNA, 2085 bp, Artificial Sequence
  A synthetic sequence
atgcggcccctgcggcctagagccgccctgctggctctcctggcttctctgctggccgctccccctgtcgcccctgccgaagccccccacctggtgcaggtggacgccgccagagccctgtggcccctgaggcggttctggcggagcaccggcttttgcccccctctgccccacagccaggccgaccagtacgtgctgtcctgggaccagcagctgaacctggcctacgtgggcgccgtgccccaccggggcatcaagcaggtgcggacccactggctgctggaactggtgaccacccggggcagcaccggcaggggcctgagctacaacttcacccacctggacggctacctggacctgctgcgggagaaccagctgctgcccggcttcgagctgatgggcagcgccagcggccacttcaccgacttcgaggacaagcagcaggtgttcgagtggaaggacctggtgtccagcctggctcgccggtacatcggcagatacggcctggcccacgtgagcaagtggaacttcgagacctggaacgagcccgaccaccacgacttcgacaacgtgagcatgaccatgcagggctttctgaactactacgacgcctgcagcgaggggctgagagccgccagccctgccctgagactgggcggacccggcgacagcttccacaccccccccagaagccccctgagctggggcctgctgcggcactgccacgacggcaccaatttcttcaccggcgaggccggcgtgcggctggactacatcagcctgcaccggaagggcgccagaagcagcatcagcatcctggaacaggaaaaggtcgtcgctcagcagatccggcagctgttccccaagttcgccgacacccccatctacaacgacgaggccgaccccctggtgggctggtctctgccccagccttggagggccgacgtgacctacgccgccatggtggtgaaggtgatcgcccagcaccagaacctgctgctggccaacaccacctccgccttcccttacgccctgctgtccaacgacaacgccttcctgagctaccacccccaccccttcgcccagcggaccctgaccgcccggttccaggtgaacaacaccagacccccccacgtgcagctgctgcggaagcccgtgctgaccgccatggggctgctggccctgctggacgaggaacagctgtgggccgaggtgtcccaggccggcaccgtgctggacagcaaccacaccgtgggcgtgctggctagcgcccacagacctcagggccctgccgatgcctggagagccgccgtgctgatctacgccagcgacgacaccagagcccaccccaaccggtccgtggccgtgaccctgcggctgagaggcgtgcctcccggccctggcctggtgtacgtgaccagatacctggacaacggcctgtgcagccccgacggcgagtggcggaggctgggcaggcccgtgttccccaccgccgagcagttccggcggatgagagccgccgaggaccccgtggccgctgcccctagacctctgcctgccggcggacggctgaccctgagacccgccctgaggctgcccagcctgctgctggtgcacgtgtgcgccaggcccgagaagcccccaggccaggtgacccggctgcgcgccctgcctctgacccagggccagctggtgctggtgtggagcgacgagcacgtgggcagcaagtgcctgtggacctacgagatccagttcagccaggacggcaaggcctacacccccgtgagccggaagcccagcaccttcaacctgttcgtgttcagccccgacacaggcgccgtgagcggcagctacagagtgcgggccctggactactgggctcgccctggccccttcagcgaccccgtgccctacctggaagtgcccgtgcccagaggccctcccagccccggcaacccccgatcgagcgctacgcgtgtcgacgaacaaaaactcatctcagaagaggatctgattgacattttggagctgcgcaagctgcgtaagcggctcctcctgcgcaagctgcgtaagcggctcctctag

SEQ ID NO: 6 - protein, 5 aa, Artificial Sequence
  A synthetic sequence
IDILE

SEQ ID NO: 7 - protein, 18 aa, Artificial Sequence
  A synthetic sequence
LRKLRKRLLLRKLRKRLL

SEQ ID NO: 8 - protein, 21 aa, Artificial Sequence
  A synthetic sequence
LGGGGSGGGGSGGGGSGGGGS

SEQ ID NO: 9 - skipped

SEQ ID NO: 10 - protein, 5 aa, Artificial Sequence
  A synthetic sequence
  SITE 1,3,4: Xaa = I, L, V, A or G
XDXXE

SEQ ID NO: 11 - protein, 5 aa, Artificial Sequence
  A synthetic sequence
  SITE 1-4: Xaa = I, L, V, A, or G
  SITE 5: Xaa = S or T
XXXXX

SEQ ID NO: 12 - protein, 5 aa, Artificial Sequence
  A synthetic sequence
GGGGS

SEQ ID NO: 13 - protein, 7 aa, Artificial Sequence
  A synthetic sequence
GSSSSSS

SEQ ID NO: 14 - protein, 21 aa, Artificial Sequence
  A synthetic sequence
LGGGGSGGGGSGGGGSGGGGS

SEQ ID NO: 15 - protein, 9 aa, Artificial Sequence
  A synthetic sequence
  SITE 1,4,5,8,9: Xaa = I, L, V, A or G
  SITE 2,3,5,6,7: Xaa = R, K, or H
XXXXXXXXX

SEQ ID NO: 16 - protein, 5 aa, Artificial Sequence
  A synthetic sequence
GGGGS

SEQ ID NO: 17 - protein, 12 aa, Artificial Sequence
  A synthetic sequence
GSAGSAAGSGEF

SEQ ID NO: 18 - protein, 9 aa, Artificial Sequence
  A synthetic sequence
LRKLRKRLL

SEQ ID NO: 19 - skipped

SEQ ID NO: 20 - protein, 15 aa, Artificial Sequence
  A synthetic sequence
GGGGSGGGGSGGGGS

SEQ ID NO: 21 - protein, 10 aa, Artificial Sequence
  A synthetic sequence
GGGGSGGGGS

SEQ ID NO: 22 - protein, 20 aa, Artificial Sequence
  A synthetic sequence
GGGGSGGGGSGGGGSGGGGS

SEQ ID NO: 23 - protein, 15 aa, Artificial Sequence
  A synthetic sequence
EAAAKEAAAKEAAAK

SEQ ID NO: 24 - protein, 556 aa, Artificial Sequence
  A synthetic sequence
MELCGLGLPRPPMLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSGSEPSGTFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLNRISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 25 - protein, 46 aa, Artificial Sequence
  A synthetic sequence
AEAAAKEAAAKEAAAKEAAAKALEAEAAAKEAAAKEAAAKEAAAKA

SEQ ID NO: 26 - protein, 36 aa, Artificial Sequence
  A synthetic sequence
AEAAAKEAAAKEAAAKALEAEAAAKEAAAKEAAAKA

SEQ ID NO: 27 - protein, 30 aa, Artificial Sequence
  A synthetic sequence
AEAAAKEAAAKALEALEAAEAAAKEAAAKA

SEQ ID NO: 28 - protein, 56 aa, Artificial Sequence
  A synthetic sequence
AEAAAKEAAAKEAAAKEAAAKEAAAKALEAEAAAKEAAAKEAAAKEAAAKEAAAKA

SEQ ID NO: 29 - skipped

SEQ ID NO: 30 - protein, 5 aa, Artificial Sequence
  A synthetic sequence
PAPAP

SEQ ID NO: 31 - protein, 12 aa, Artificial Sequence
  A synthetic sequence
AEAAAKEAAAKA

SEQ ID NO: 32 - protein, 20 aa, Artificial Sequence
  A synthetic sequence
GGGGSGGGGSGGGGSGGGGS

SEQ ID NO: 33 - protein, 17 aa, Artificial Sequence
  A synthetic sequence
VSQTSKLTRAETVFPDV

SEQ ID NO: 34 - protein, 6 aa, Artificial Sequence
  A synthetic sequence
PLGLWA

SEQ ID NO: 35 - protein, 6 aa, Artificial Sequence
  A synthetic sequence
RVLAEA

SEQ ID NO: 36 - protein, 10 aa, Artificial Sequence
  A synthetic sequence
EDVVCCSMSY

SEQ ID NO: 37 - protein, 8 aa, Artificial Sequence
  A synthetic sequence
GGIEGRGS

SEQ ID NO: 38 - protein, 10 aa, Artificial Sequence
  A synthetic sequence
TRHRQPRGWE

SEQ ID NO: 39 - protein, 13 aa, Artificial Sequence
  A synthetic sequence
RRRRRRRRRGFLG

SEQ ID NO: 40 - protein, 10 aa, Artificial Sequence
  A synthetic sequence
AGNRVRRSVG

SEQ ID NO: 41 - skipped

SEQ ID NO: 42 - protein, 5 aa, Artificial Sequence
  A synthetic sequence
  SITE 1,3,4: Xaa = I, L, V, A or G
XDXXE

SEQ ID NO: 43 - protein, 5 aa, Artificial Sequence
  A synthetic sequence
IDILE

SEQ ID NO: 44 - protein, 5 aa, Artificial Sequence
  A synthetic sequence
  SITE 1-4: Xaa = I, L, V, A or G
  SITE 5: Xaa = S or T
XXXXX

SEQ ID NO: 45-46 - skipped (2 entries)

SEQ ID NO: 47 - protein, 21 aa, Artificial Sequence
  A synthetic sequence
LGGGGSGGGGSGGGGSGGGGS

SEQ ID NO: 48 - protein, 5 aa, Artificial Sequence
  A synthetic sequence
  SITE 2,5: Xaa = D, E, R, K, N or Q
  SITE 1,3,4: Xaa = I, L, V, A or G
XXXXX

SEQ ID NO: 49 - protein, 9 aa, Artificial Sequence
  A synthetic sequence
  SITE 1,4,5,8,9: Xaa = I, L, V, A or G
  SITE 2,3,5,6,7: Xaa = R, K or H
XXXXXXXXX

SEQ ID NO: 50 - protein, 9 aa, Artificial Sequence
  A synthetic sequence
LRKLRKRLL

SEQ ID NO: 51 - protein, 8 aa, Artificial Sequence
  A synthetic sequence
RSSATRVD